(19) United States
(12) Patent Application Publication
(10) Pub. No.: US 2024/0318110 A1
(43) Pub. Date: Sep. 26, 2024

(54) MODULES AND INSTRUMENTS FOR AUTOMATED NUCLEIC ACID-GUIDED NUCLEASE EDITING IN MAMMALIAN CELLS USING MICROCARRIERS

(71) Applicant: INSCRIPTA, INC., Boulder, CO (US)

(72) Inventors: Andrea BRYAN, Boulder, CO (US); Bruce CHABANSKY, Boulder, CO (US); David STUMBO, Boulder, CO (US); Eric SMITH, Boulder, CO (US); Jorge BERNATE, Boulder, CO (US); Diane BRANNIGAN, Boulder, CO (US)

(73) Assignee: INSCRIPTA, INC., Boulder, CO (US)

(21) Appl. No.: 18/681,313

(22) PCT Filed: Aug. 4, 2022
(86) PCT No.: PCT/US2022/039368

§ 371 Date: Feb. 5, 2024

Related U.S. Application Data
(60) Provisional application No. 63/230,041, filed on Aug. 5, 2021.

(51) Int. Cl. C12M 1/00; C12M 1/06; C12M 1/12; C12M 1/34

(52) U.S. Cl. CPC C12M 23/58; C12M 23/38; C12M 25/02; C12M 27/06; C12M 29/04; C12M 41/12; C12M 41/36

(57) ABSTRACT

This invention relates to modules and automated, integrated, end-to-end closed instruments for automated mammalian cell growth and mammalian cell transfection followed by nucleic acid-guided nuclease editing in live mammalian cells.

### RELATED APPLICATIONS

This International PCT Application claims the benefit of priority to U.S. Provisional Application No. 63/230,041 filed 5 Aug. 2021 and is incorporated herein by reference.

### FIELD OF THE INVENTION

This invention relates to modules and automated end-to-end instruments for automated mammalian cell growth and mammalian cell transfection followed by nucleic acid-guided nuclease editing in live mammalian cells.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently various nucleases have been identified that allow manipulation of gene sequence; hence, gene function. The nucleases include nucleic acid-guided nucleases, which enable researchers to generate permanent edits in live cells. Editing efficiencies frequently correlate with the concentration of guide RNAs (gRNAs) and repair templates (e.g., repair templates or homology arms) in the cell, particularly in mammalian cells. That is, the higher the concentration of gRNA and repair templates, the better the editing efficiency. Further, it is desirable to be able to perform many different edits in a population of mammalian cells simultaneously and to do so in an automated fashion, minimizing manual or hands-on cell manipulation.

There is thus a need in the art of nucleic acid-guided nuclease gene editing for improved modules and instrumentation for increasing nucleic acid-guided nuclease editing efficiency and throughput in live mammalian cells, particularly in an end-to-end, closed and fully-automated instrument. The present invention satisfies this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to modules and multi-module automated instrumentation for making edits in a mammalian genome. Efficient editing requires many excess copies of editing cassettes—comprising a gRNA and a repair template (e.g., repair template or homology arm)—in the cell nucleus. In order to perform highly-multiplexed editing in a single reaction, it is necessary to co-localize the cells with many clonal copies of each editing cassette. Thus, the present disclosure entails making “reagent bundles” comprising many (hundreds of thousands to millions) clonal copies of an editing cassette, growing and passaging mammalian cells, and delivering and co-localizing the reagent bundles with the mammalian cells such that the editing cassettes edit the cells and the edited cells continue to grow. In doing so, it is preferable to use a fully-automated, end-to-end closed instrument that does not require human intervention throughout the cell growth, passaging and editing cycle to provide consistent results in the workflow thereby enhancing uniformity of processing between “batches” and maintaining sample integrity.

Thus, in some embodiments there is provided an integrated instrument for growing, passaging and editing cells comprising: a bioreactor comprising: a growth vessel comprising a tapered main body, a lid assembly comprising ports, at least one driving impeller, and an impeller shaft, wherein there is at least two liquid ports, wherein at least one of the liquid ports comprises a filtered sipper; at least one gas-in port; at least one gas-out port; at least one rupture disc; and at least one sensor port; and wherein the lid assembly makes an air-tight fitting on the tapered main body; and a bioreactor stand assembly comprising a frame, a stand main body disposed in the frame, wherein the stand main body accommodates the tapered main body of the growth vessel during operation, and wherein the stand main body comprises a heating element to heat the tapered main body; and a cell corral comprising a main body configured to store cells and fluidically coupled to the bioreactor tapered main body via the liquid port comprising the filtered sipper.

In some aspects of this embodiment, the lid assembly further comprises a motor integration port for a motor to control the impeller, and in some aspects, the motor integration port comprises a magnetic connection between the bioreactor tapered main body and a motor. In some aspects, the bioreactor comprises a second impeller. In some aspects of this embodiment, there are separate liquid-in and liquid-out ports, and in other aspects of this embodiment, the liquid ports may serve as both liquid-in and liquid-out ports; that is, a liquid port may be a dedicated in or out port, or may serve both as an in port and an out port.

In some aspects of either of these embodiments, the at least one sensor port in the lid assembly is configured to accommodate a monitor capacitance of the cells and medium in the tapered main body of the growth vessel; a sensor to measure dissolved O₂ concentration of the cells and medium in the tapered main body of the growth vessel; a sensor to measure dissolved CO₂ of the cells and medium in the tapered main body of the growth vessel; a sensor to measure pH of the cells and medium in the tapered main body of the growth vessel; a sensor to measure lactate concentration of the cells and medium in the tapered main body of the growth vessel; a sensor to measure glucose concentration of the cells and medium in the tapered main body of the growth vessel; a sensor to measure biomass of the cells and medium in the tapered main body of the growth vessel; or a sensor to measure optical density of the cells and medium in the tapered main body of the growth vessel, and in some embodiments, there are at least two, at least three or at least four sensor ports in the lid assembly each configured to monitor capacitance of the cells and medium in the tapered main body of the growth vessel; a sensor to measure dissolved O₂ concentration of the cells and medium in the tapered main body of the growth vessel; a sensor to measure dissolved CO₂ of the cells and medium in the tapered main body of the growth vessel; a sensor to measure pH of the cells and medium in the tapered main body of the growth vessel; a sensor to measure lactate concentration of the cells and medium in the tapered main body of the growth vessel; a sensor to measure glucose concentration of the cells and medium in the tapered main body of the growth vessel; a sensor to measure biomass of the cells and medium in the tapered main body of the growth vessel; or a sensor to measure optical density of the cells and medium in the tapered main body of the growth vessel.

In some aspects of these embodiments, the lid assembly further comprises a temperature probe, and in some aspects, the lid assembly further comprises a camera port. In some aspects the heating element of the stand main body is a heat jacket, and in some aspects, the heat jacket comprises LED lights and may also comprise a camera port.

In some aspects of these embodiments, the cell corral comprises a heat jacket.

Also provided is a method of growing cells and passaging the cells in an integrated instrument, comprising the steps of: providing an integrated instrument comprising: a bioreactor comprising a growth vessel comprising a tapered main body, a lid assembly comprising ports, at least one driving impeller, and an impeller shaft, wherein there is at least two liquid ports, wherein at least one of the liquid ports comprises a filtered sipper and at least one of the liquid ports comprises a non-filtered sipper and at least one liquid port is both a liquid-out port and a liquid-in port; at least one gas-in port; at least one gas-out port; at least one rupture disc; and at least one sensor port; and wherein the lid assembly makes an air-tight fitting on the tapered main body; and a bioreactor stand assembly comprising a frame, a stand main body disposed in the frame, wherein the stand main body accommodates the tapered main body of the growth vessel during operation, and wherein the stand main body comprises a heating element to heat the tapered main body; and a cell corral comprising a main body configured to store cells and fluidically coupled to the bioreactor tapered main body via the liquid port comprising the filtered sipper; providing microcarriers comprising a cell adhesion agent in cell growth medium to the growth vessel; providing cells to the growth vessel; allowing the cells to adhere to the microcarriers; growing the cells on the microcarriers; dissociating the cells from the microcarriers; allowing the microcarriers to settle on a bottom of the growth vessel; aspirating the cells into the cell corral via the liquid port comprising the filtered sipper; aspirating the microcarriers into waste via the liquid port comprising the non-filtered sipper; washing the growth vessel; adding fresh medium and microcarriers to the growth vessel, wherein the microcarriers comprise a cell adhesion agent; transferring the cells in the cell corral to the growth vessel via the liquid port comprising the filtered sipper; and allowing the cells to adhere to the microcarriers.

Some aspects of this method embodiment, further comprise the steps of: growing the cells on the microcarriers; dissociating the cells from the microcarriers; allowing the microcarriers to settle on a bottom of the growth vessel; aspirating the cells into the cell corral via the liquid port comprising the filtered sipper; aspirating the microcarriers into waste via the liquid port comprising the non-filtered sipper; washing the growth vessel; providing cell growth medium and reagent bundle microcarriers to the tapered main body of the growth vessel, wherein each reagent bundle microcarrier comprises clonal copies of editing cassettes, a selection marker, a coding sequence for a nucleic acid-guided nuclease and a lipofection agent; transferring the cells from the cell corral to the growth vessel via the liquid port comprising the sipper; allowing the cells to attach to and grow on the reagent bundle microcarriers; providing conditions for the editing cassettes to transfect the cells; selecting for transfected cells via the selection marker; dissociating the cells from the reagent bundle microcarriers; allowing the reagent bundle microcarriers to settle in the bottom of the growth vessel; and aspirating the cells into the cell corral via the liquid port comprising the filtered sipper. In next steps, the reagent bundle microcarriers are removed from the growth vessel, the growth vessel is washed, microcarriers and medium are dispensed into the growth vessel, and the cells are transferred from the cell corral via the liquid port comprising the filter.

In some aspects of the method embodiments, the tapered main body of the growth vessel accommodates cell culture volumes of 25 ml to 500 ml. In some aspects, during cell growth impeller revolutions per minute is approximately 40-80 rpm, and in some aspects during cell detachment impeller revolutions per minute is approximately 1200-2700 rpm. In some aspects of either of these embodiments, the tapered main body is optically transparent and in some aspects, the tapered main body is optically transparent in UV and IR ranges.

In some aspects of these method embodiments, a chemical agent is added to the tapered main body of the growth vessel to aid in detaching the cells, and in some aspects, the chemical agent is hemagglutinin, collagenase, dispase or trypsin.

In some aspects of these embodiments, the nuclease is provided as a protein and in other aspects, the nuclease is provided as a nucleic acid coding sequence under control of a promoter.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A depicts an exemplary workflow employing microcarrier-partitioned delivery for editing mammalian cells grown in suspension. FIG. 1B depicts an option for growing, passaging, transfecting and editing iPSCs involving sequential transfection of editing cassettes and nuclease.

FIG. 1C depicts an exemplary workflow employing microcarrier-partitioned delivery for editing mammalian cells. FIG. 1D depicts an alternative workflow employing microcarrier-partitioned delivery for editing mammalian cells. FIG. 1E depicts an exemplary architecture for editing cassettes to be delivered as RBMCs (reagent bundle microcarriers or reagent vehicle microcarriers (rvMCs).

FIGS. 2A-2J depict various components of exemplary embodiments of a bioreactor useful for growing and transducing mammalian cells by the methods described herein. FIGS. 2K-1 and 2K-2 depict an exemplary fluidic diagram for the bioreactor described in relation to FIGS. 2A-2J. FIG. 2L depicts an exemplary control system block diagram for the bioreactor described in relation to FIGS. 2A-2G.

FIGS. 3A-3D depict various components of exemplary embodiments of a “cell corral” companion vessel for the exemplary bioreactors shown in FIGS. 2A-2J.

FIG. 4 is a simplified workflow and two photographs of fluorescent cells on microcarriers demonstrating the integrity of nucleic acids loaded on laminin-coated microcarriers when mixed.

FIG. 5 is a simplified workflow and a photograph of fluorescent cells demonstrating that LNPs absorb onto L521 microcarriers and remain functional for transfection after washing.

FIG. 6 is a bar graph showing data demonstrating that microcarrier fidelity is tunable by the adsorption protocol used.

FIG. 7 is a bar graph showing data demonstrating that green-to-blue editing on LNPsMCs is equivalent to reverse transcription on a tissue culture plate.

FIGS. 8A and 8B are graphs demonstrating that the materials comprising the components of the bioreactor are biocompatible.

FIG. 9 comprises three graphs demonstrating that iPSC culture and cell expansion in the bioreactor described herein is comparable to cell culture and expansion in a CORNING® spinner flask and in a traditional cell culture plate.

FIG. 10 is a graph demonstrating that media exchange at ˜200 ml/minute does not impact cell growth.

FIG. 11 is a series of four graphs demonstrating that up to five rounds of impeller shear is tolerated by iPSCs with no negative effects on re-seeding.

FIG. 12 shows a workflow at top right, a table reporting percent efficiency at various steps in the workflow at center, and a graph showing the replicates measuring the percent efficiency at various steps in the workflow at bottom.

FIG. 13 is a graph showing that cell seeding and expansion are both unaffected by the impeller-shear based passaging protocol.

FIG. 14 at top are histograms showing the fluorescent expression distribution measured via flow cytometry of the cell population for individual stemness marker expression. FIG. 14 at bottom left is a bar graph showing a stemness panel (FACS % positive) for cells in the bioreactor described herein, on laminin plates and on MATRIGEL® plates (CORNING® BIOCOAT™ MATRIGEL® 6-well plates (Corning, Inc., Glendale, AZ)). FIG. 14 at bottom right is a bar graph showing a stemness panel (FACS median fluorescence) for cells in the bioreactor described herein, on laminin plates and on MATRIGEL® plates (CORNING® BIOCOAT™ MATRIGEL® 6-well plates (Corning, Inc., Glendale, AZ)).

FIG. 15A-15F shows a series of panels, both % positive and median fluorescence, demonstrating that iPSCs grown in the bioreactor described herein maintain differentiation potential comparable to iPSCs cultured on laminin plates and in MATRIGEL® plates (CORNING® BIOCOAT™ MATRIGEL® 6-well plates (Corning, Inc., Glendale, AZ)).

It should be understood that the drawings are not necessarily to scale, and that like reference numbers refer to like features.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis and hybridization and ligation of polynucleotides. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3rd Ed., W. H. Freeman Pub., New York, N.Y.; Viral Vectors (Kaplift & Loewy, eds., Academic Press 1995); all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza & Atala, eds., Academic Press 2013); and Handbook of Stem Cells, (Atala & Lanza, eds., Academic Press 2012). CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The terms “editing cassette”, “CREATE cassette”, “CREATE editing cassette”, “CREATE fusion editing cassette” or “CFE editing cassette” refers to a nucleic acid molecule comprising a coding sequence for transcription of a guide nucleic acid or gRNA covalently linked to a coding sequence for transcription of a repair template.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The term “microcarrier” refers to nonporous, microporous, and macroporous microcarriers comprising natural organic materials such as, e.g., gelatin, collagen, alginate, agarose, chitosan, and cellulose; synthetic polymeric materials such as, e.g., polystyrene, polyacrylates such as polyacrylamide, polyamidoamine (PAMAM), polyethylene oxide (PEO/PEG), poly(N-isopropylacrylamide) (PNIPAM), polycaprolactone (PCL), polylactic acid (PLA), and polyglycolic acid (PGA); inorganic materials such as, e.g., silica, silicon, mica, quartz and silicone; as well as mixtures of natural, polymeric materials, crossed-linked materials, and inorganic materials etc., on which animal cells can grow. The terms “reagent vehicle microcarrier” (i.e., rvMC), “reagent bundle microcarrier” (i.e., RBMC), “lipofectamine and nucleic acid microcarrier” (i.e., LNPsMC) refer to a microcarrier with a payload; as used herein a payload of nucleic acids, proteins or protein complexes to be transfected into cells and/or transfection reagents and/or cells.

“Nucleic acid-guided editing components” refers to one, some, or all of a nucleic acid-guided nuclease or nickase fusion enzyme, a guide nucleic acid and a repair template.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

As used herein, a “partition” is an isolated region (e.g., a feature surrounded by an interstitial region) on a substrate, an isolate depression (e.g., a well) on a substrate, a droplet, or a microcarrier. Partitions are used, in relation to the present disclosure, to separate a plurality to many different nucleic acids (e.g., editing cassettes).

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible.

As used herein the terms “repair template” or “donor nucleic acid” or “donor DNA” or “homology arm” refer to 1) nucleic acid that is designed to introduce a DNA sequence modification (insertion, deletion, substitution) into a locus by homologous recombination using nucleic acid-guided nucleases, or 2) a nucleic acid that serves as a template (including a desired edit) to be incorporated into target DNA by reverse transcriptase in a CREATE fusion editing (CFE) system. For homology-directed repair, the repair template must have sufficient homology to the regions flanking the “cut site” or the site to be edited in the genomic target sequence. For template-directed repair, the repair template has homology to the genomic target sequence except at the position of the desired edit although synonymous edits may be present in the homologous (e.g., non-edit) regions. The length of the repair template(s) will depend on, e.g., the type and size of the modification being made. In many instances and preferably, the repair template will have two regions of sequence homology (e.g., two homology arms) complementary to the genomic target locus flanking the locus of the desired edit in the genomic target locus. Typically, an “edit region” or “edit locus” or “DNA sequence modification” region—the nucleic acid modification that one desires to be introduced into a genome target locus in a cell (e.g., the desired edit)—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTAI; conjugates glutathione to the stem cell selective alkylator busulfan; chemoprotective selectable marker in CD34+ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” and the like refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The terms “transformation”, “transfection” and “transduction” are used interchangeably herein to refer to the process of introducing exogenous DNA into cells.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In some embodiments, a coding sequence for a nucleic acid-guided nuclease is provided in a vector, referred to as an “engine vector.” In some embodiments, the editing cassette may be provided in a vector, referred to as an “editing vector.” In some embodiments, the coding sequence for the nucleic acid-guided nuclease and the editing cassette are provided in the same vector.

A “viral vector” as used herein is a recombinantly produced virus or viral particle that comprises an editing cassette to be delivered into a host cell. Examples of viral vectors include retroviral vectors, lentiviral vectors, adenovirus vectors, adeno-associated virus vectors, alphavirus vectors and the like.

### Nuclease-Directed Genome Editing Generally

The modules and integrated instruments described herein are employed to allow one to perform nucleic acid nuclease-directed genome editing to introduce desired edits to a population of live mammalian cells in a closed, end-to-end automated instrument. The modules, instruments and methods entail employing reagent bundle microcarriers (RBMCs) comprising many clonal (e.g., identical) copies of editing cassettes—that is, the editing cassettes on a single microcarrier will be clonal copies of one another—followed by co-localizing the RBMCs with live mammalian cells to effect editing of the genome of the mammalian cells by the editing cassettes. The RBMCs are manufactured off-instrument and are co-located with the cells on-instrument for automated cell editing.

Generally, a nucleic acid-guided nuclease or nickase fusion complexed with an appropriate synthetic guide nucleic acid in a cell can cut the genome of the cell at a desired location. The guide nucleic acid helps the nucleic acid-guided nuclease or nickase fusion recognize and cut the DNA at a specific target sequence. By manipulating the nucleotide sequence of the guide nucleic acid, the nucleic acid-guided nuclease or nickase fusion may be programmed to target any DNA sequence for cleavage as long as an appropriate protospacer adjacent motif (PAM) is nearby. In certain aspects, the nucleic acid-guided nuclease or nickase fusion editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion.

In general, a guide nucleic acid (e.g., gRNA) complexes with a compatible nucleic acid-guided nuclease or nickase fusion enzyme and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. Preferably and typically, the guide nucleic acid comprises RNA and the gRNA is encoded by a DNA sequence on an editing cassette along with the coding sequence for a repair template. Covalently linking the gRNA and repair template allows one to scale up the number of edits that can be made in a population of cells tremendously. Methods and compositions for designing and synthesizing editing cassettes (e.g., CREATE cassettes) are described in U.S. Pat. Nos. 10,240,167; 10,266,849; 9,982,278; 10,351,877; 10,364,442; 10,435,715; 10,669,559; 10,711,284; 10,731,180, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion enzyme to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease or gRNA/nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, in the case of mammalian cells the target sequence is typically a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA). The proto-spacer mutation (PAM) is a short nucleotide sequence recognized by the gRNA/nuclease complex. The precise preferred PAM sequence and length requirements for different nucleic acid-guided nucleases or nickase fusions vary; however, PAMs typically are 2-7 base-pair sequences adjacent or in proximity to the target sequence and, depending on the nuclease or nickase, can be 5′ or 3′ to the target sequence.

In most embodiments, genome editing of a cellular target sequence both introduces a desired DNA change (i.e., the desired edit) to a cellular target sequence, e.g., the genomic DNA of a cell, and removes, mutates, or renders inactive a proto-spacer/spacer mutation (PAM) region in the cellular target sequence (e.g., thereby rendering the target site immune to further nuclease binding). Rendering the PAM and/or spacer at the cellular target sequence inactive precludes additional editing of the cell genome at that cellular target sequence, e.g., upon subsequent exposure to a nucleic acid-guided nuclease or nickase fusion complexed with a synthetic guide nucleic acid in later rounds of editing. Thus, cells having the desired cellular target sequence edit and an altered PAM or spacer can be selected for by using a nucleic acid-guided nuclease or nickase fusion complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM or spacer alteration will not be cut, as these edited cells no longer contain the necessary PAM site and will continue to grow and propagate.

As for the nuclease or nickase fusion component of the nucleic acid-guided nuclease editing system, a polynucleotide sequence encoding the nucleic acid-guided nuclease or nickase fusion can be codon optimized for expression in particular cell types, such as bacterial, yeast, and, here, mammalian cells. The choice of the nucleic acid-guided nuclease or nickase fusion to be employed depends on many factors, such as what type of edit is to be made in the target sequence and whether an appropriate PAM is located close to the desired target sequence. Nucleases of use in the methods described herein include but are not limited to Cas 9, Cas 12/Cpf1, MAD2, or MAD7, MAD 2007 or other MADzymes and MADzyme systems (see U.S. Pat. Nos. 9,982,279; 10,337,028; 10,435,714; 10,011,849; 10,626,416; 10,604,746; 10,665,114; 10,640,754; 10,876,102; 10,883,077; 10,704,033; 10,745,678; 10,724,021; 10,767,169; and 10,870,761 for sequences and other details related to engineered and naturally-occurring MADzymes). Nickase fusion enzymes typically comprise a CRISPR nucleic acid-guided nuclease engineered to cut one DNA strand in the target DNA rather than making a double-stranded cut, and the nickase portion is fused to a reverse transcriptase. For more information on nickases and nickase fusion editing see U.S. Pat. No. 10,689,669 and Ser. Nos. 16/740,418; 16/740,420 and 16/740,421, both filed 11 Jan. 2020. A coding sequence for a desired nuclease or nickase fusion may be on an “engine vector” along with other desired sequences such as a selective marker or may be transfected into a cell as a protein or ribonucleoprotein (“RNP”) complex.

Another component of the nucleic acid-guided nuclease or nickase fusion system is the repair template comprising homology to the cellular target sequence. In some exemplary embodiments, the repair template is in the same editing cassette as (e.g., is covalently-linked to) the guide nucleic acid and typically is under the control of the same promoter as the gRNA (that is, a single promoter driving the transcription of both the editing gRNA and the repair template). The repair template is designed to serve as a template for homologous recombination with a cellular target sequence cleaved by the nucleic acid-guided nuclease or serve as the template for template-directed repair via the nickase fusion, as a part of the gRNA/nuclease complex. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Pat. No. 10,711,284.

In certain preferred aspects, the repair template can be provided as an oligonucleotide of between 20-300 nucleotides, more preferably between 50-250 nucleotides. As described infra, the repair template comprises a region that is complementary to a portion of the cellular target sequence. When optimally aligned, the repair template overlaps with (is complementary to) the cellular target sequence by, e.g., about as few as 4 (in the case of nickase fusions) and as many as 20, 25, 30, 35, 40, 50, 60, 70, 80, 90 or more nucleotides (in the case of nucleases). The repair template comprises a region complementary to the cellular target sequence flanking the edit locus or difference between the repair template and the cellular target sequence. The desired edit may comprise an insertion, deletion, modification, or any combination thereof compared to the cellular target sequence.

As described in relation to the gRNA, the repair template may be provided as part of a rationally-designed editing cassette along with a promoter to drive transcription of both the gRNA and repair template. As described below, the editing cassette may be provided as a linear editing cassette, or the editing cassette may be inserted into an editing vector. Moreover, there may be more than one, e.g., two, three, four, or more editing gRNA/repair template pairs rationally-designed editing cassettes linked to one another in a linear “compound cassette” or inserted into an editing vector; alternatively, a single rationally-designed editing cassette may comprise two to several editing gRNA/repair template pairs, where each editing gRNA is under the control of separate different promoters, separate promoters, or where all gRNAs/repair template pairs are under the control of a single promoter. In some embodiments the promoter driving transcription of the editing gRNA and the repair template (or driving more than one editing gRNA/repair template pair) is an inducible promoter. In many if not most embodiments of the compositions, methods, modules and instruments described herein, the editing cassettes make up a collection or library editing gRNAs and of repair templates representing, e.g., gene-wide or genome-wide libraries of editing gRNAs and repair templates.

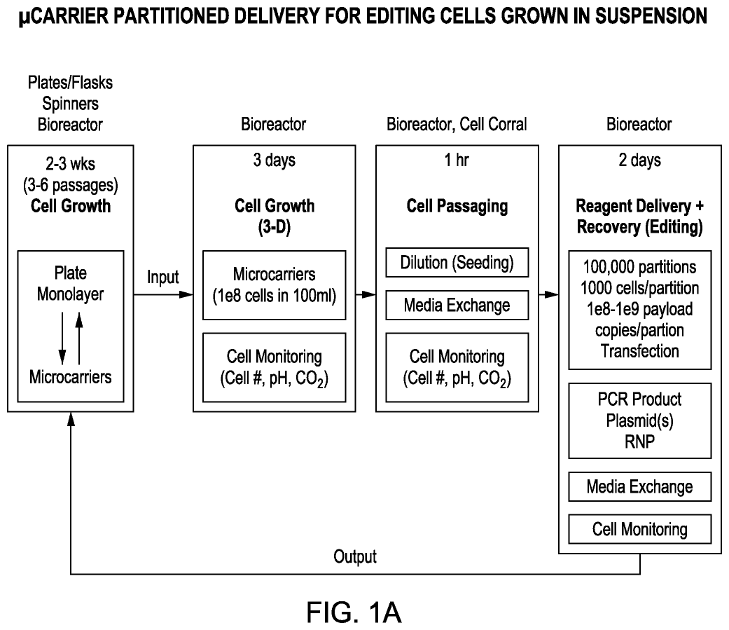

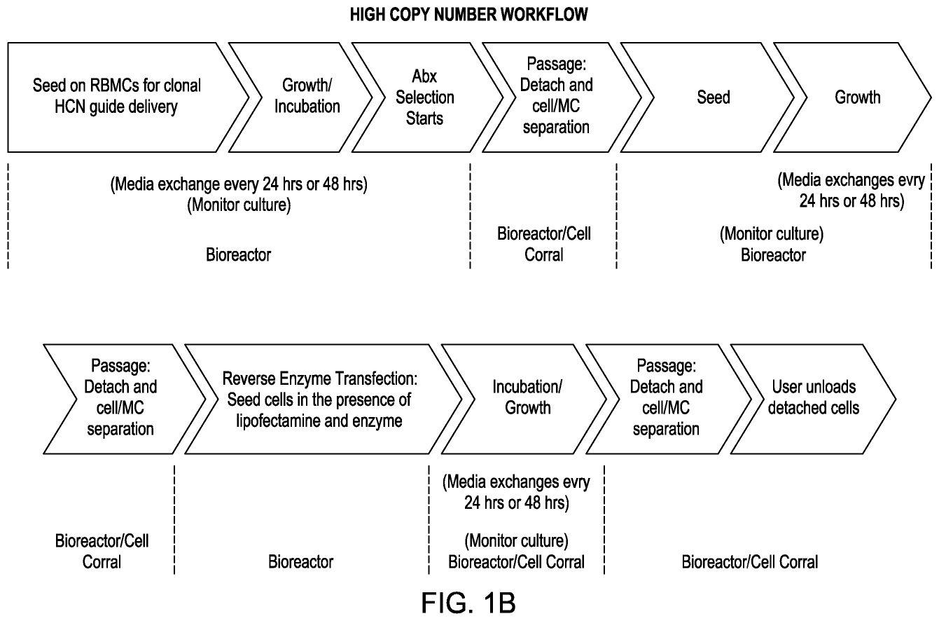

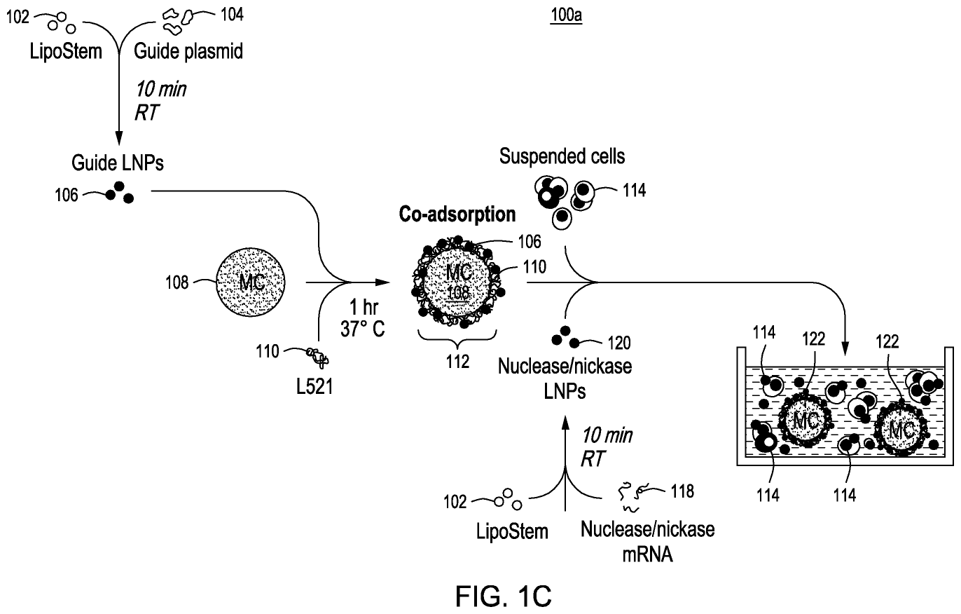

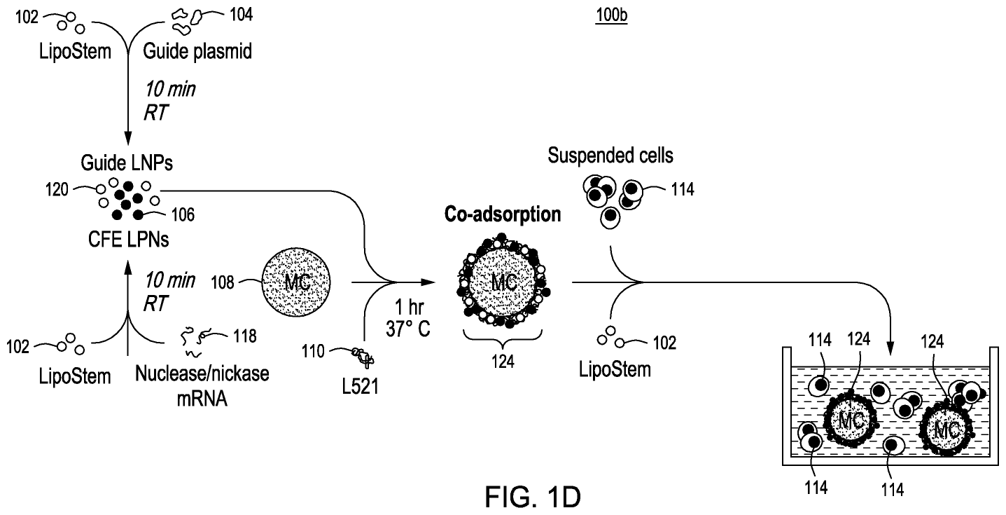

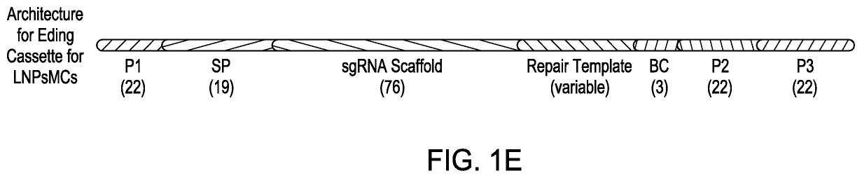

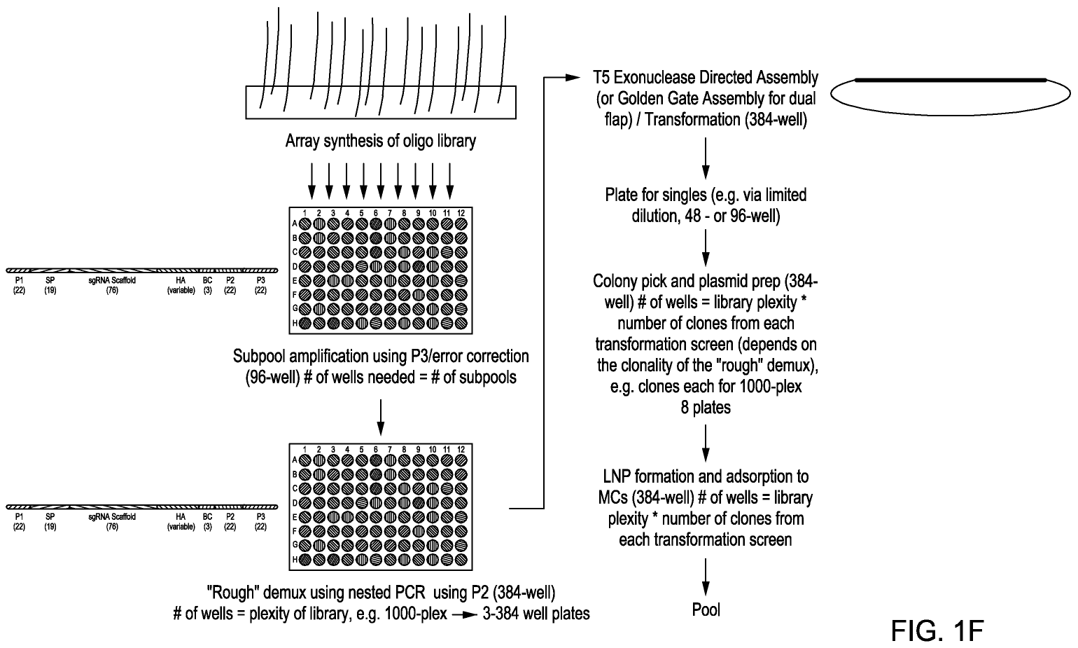

In addition to the repair template, the editing cassettes comprise one or more primer binding sites to allow for PCR amplification of the editing cassettes. The primer binding sites are used to amplify the editing cassette by using oligonucleotide primers as described infra (see, e.g., FIG. 1E), and may be biotinylated or otherwise labeled. In addition, the editing cassette may comprise a barcode. A barcode is a unique DNA sequence that corresponds to the repair template sequence such that the barcode serves as a proxy to identify the edit made to the corresponding cellular target sequence. The barcode typically comprises four or more nucleotides. Also, in preferred embodiments, an editing cassette or editing vector or engine vector further comprises one or more nuclear localization sequences (NLSs), such as about or more than about 1, 2, 3, 4, 5, 6, 7, 8, 9, 10, or more NLSs.

### Mammalian Cell Growth, Reagent Bundle Creation Transformation and Editing

In the present methods, mammalian cells are often grown in culture off-instrument for several passages before entry into the closed, end-to-end automated, growth and editing process. Cell culture is the process by which cells are grown under controlled conditions, almost always outside the cell's natural environment. For mammalian cells, culture conditions typically vary somewhat for each cell type but generally include a medium and additives that supply essential nutrients such as amino acids, carbohydrates, vitamins, minerals, growth factors, hormones, and gases such as, e.g., O₂ and CO₂. In addition to providing nutrients, the medium typically regulates the physio-chemical environment via a pH buffer, and most cells are grown at 37° C. Many mammalian cells require or prefer a surface or artificial substrate on which to grow (e.g., adherent cells), whereas other cells such as hematopoietic cells and some adherent cells can be grown in or adapted to grow in suspension. Adherent cells often are grown in 2D monolayer cultures in petri dishes or flasks, but some adherent cells can grow in suspension cultures to higher density than would be possible in 2D cultures. “Passage” generally refers to the process of transferring a small number of cells to a fresh substrate with fresh medium, or, in the case of suspension cultures, transferring a small volume of the culture to a larger volume of medium.

Mammalian cells include primary cells, which are cultured directly from a tissue and typically have a limited lifespan in culture, including T cells and NK cells; established or immortalized cell lines, which have acquired the ability to proliferate indefinitely either through random mutation or deliberate modification such as by expression of the telomerase gene; and stem cells, of which there are undifferentiated stem cells or partly-differentiated stem cells that can both differentiate into various types of cells and divide indefinitely to produce more of the same stem cells.

Primary cells can be isolated from virtually any tissue. Immortalized cell lines can be created or may be well-known, established cell lines such as human cell lines DU145 (derived from prostate cancer cells); H295R (derived from adrenocortical cancer cells); HeLa (derived from cervical cancer cells); KBM-7 (derived from chronic myelogenous leukemia cells); LNCaP (derived from prostate cancer cells); MCF-7 (derived from breast cancer cells); MDA-MB-468 (derived from breast cancer cells); PC3 (derived from prostate cancer cells); SaOS-2 (derived from bone cancer cells); SH-SY5Y (derived from neuroblastoma cells); HEK293 (derived from human embryonic kidney cells): T-047D (derived from breast cancer cells); TH-1 (derived from acute myeloid leukemia cells); U87 (derived from glioblastoma cells); and the National Cancer Institute's 60 cancer line panel NCI60; and other immortalized mammalian cell lines such as Vero cells (derived from African green monkey kidney epithelial cells); the mouse line MC3T3; rat lines GH3 (derived from pituitary tumor cells) and PC12 (derived from pheochromocytoma cells); and canine MDCK cells (derived from kidney epithelial cells).

Stem cells are of particular interest in the methods and compositions described herein. Generally speaking, there are three general types of mammalian stem cells: adult stem cells (ASCs), which are undifferentiated cells found living within specific differentiated tissues including hematopoietic, mesenchymal, neural, and epithelial stem cells; embryonic stem cells (ESCs), which in humans are isolated from a blastocyst typically 3-5 days following fertilization and which are capable of generating all the specialized tissues that make up the human body; and induced pluripotent stem cells (iPSCs), which are adult stem cells that are created using genetic reprogramming with, e.g., protein transcription factors.

Once the cells of choice have been grown and passaged several times-in most embodiments off-instrument-in a first step the mammalian cells that are to be edited are transferred to an automated instrument where the cells are grown in cell culture and the growth of the cells is monitored. Growth modules may include a rotating growth module (see, e.g., U.S. Pat. Nos. 10,435,662; 10,443,031; 10,590,375; 10,717,959; and 10,883,095), a tangential filtration module (see, e.g., Ser. Nos. 16/516,701 and 16/798,302) or a bioreactor, where exemplary embodiments of a bioreactor are described in detail infra. Moreover, these growth modules may be used for the transfection or reverse transfection steps performed to initiate editing. Monitoring is usually performed by imaging the cells as described infra and/or by, e.g., measuring pH of the medium using a medium comprising a pH indicator or pH probe. As opposed to 2D culture of cells as described above, the present methods envision culturing the cells in suspension. Growing cells in suspension can be effected in various configurations. Adherent cells that typically are grown in 2D cultures when grown in suspension often aggregate into “clumps.” For example, some iPSCs grow well as aggregates in suspension, and are most healthy growing in aggregates of 50-300 microns in size, starting off as smaller aggregates 30-50 microns in size. iPSCs are typically grown in culture 3-5 days between passaging and the larger aggregates are broken into smaller aggregates by filtering them, e.g., through a cell strainer (e.g., a sieve or frit) with a 37 micron filter. The iPSCs can grow indefinitely in 3D aggregates as long as they are passaged into smaller aggregates when the aggregates become approximately 300-400 microns in size.

An alternative to growing cells in 3D aggregates and in a preferred embodiment is growing cells on microcarriers. Generally, microcarriers are nonporous (comprising pore sizes range from 0-20 nm), microporous (comprising pore sizes range from 20 nm-1 micron), and macroporous (comprising pore sizes range from 1-50 microns). Microcarriers may be fabricated from natural organic materials such as, e.g., gelatin, collagen, alginate, agarose, chitosan, and cellulose; biocompatible synthetic polymeric materials such as, e.g., polystyrene, polyacrylates such as polyacrylamide, polyamidoamine (PAMAM), polyethylene oxide (PEO/PEG), poly(N-isopropylacrylamide) (PNIPAM), polycaprolactone (PCL), polylactic acid (PLA), and polyglycolic acid (PGA); inorganic materials such as, e.g., silica, silicon, mica, quartz and silicone; as well as mixtures of natural, polymeric materials, cross-linked polymeric materials, and inorganic materials etc. on which animal cells can grow. Microcarriers useful in the methods herein typically range in size from 30-1200 microns in diameter and more typically range in size from 40-200 or from 50-150 microns in diameter.

Finally, another option for growing mammalian cells for editing in the compositions, methods, modules and automated instruments described herein is growing single cells in suspension using a specialized medium such as that developed by ACCELLTA™ (Haifa, Israel). Cells grown in this medium must be adapted to this process over many cell passages; however, once adapted the cells can be grown to a density of >40 million cells/ml and expanded 50-100× in approximately a week, depending on cell type.

The cells grown off-instrument or, more typically, in a growth module of the automated instrument as well as reagents needed for cell growth, nucleic acid amplification, cell transfection (e.g., the RBMCs or rvMCs), cell editing and enrichment may be provided in a reagent cartridge, particularly in a closed, fully-automated instrument as described herein. The cells and reagents may be moved from the reagent cartridge and between modules by a robotic liquid handling system which may include a gantry. As an example, the robotic liquid handling system may include an automated liquid handling system such as those manufactured by Tecan Group Ltd. of Mannedorf, Switzerland, Hamilton Company of Reno, NV (see, e.g., WO2018015544A1 to Ott, entitled “Pipetting device, fluid processing system and method for operating a fluid processing system”), or Beckman Coulter, Inc. of Fort Collins, CO. (see, e.g., US20160018427A1 to Striebl et al., entitled “Methods and systems for tube inspection and liquid level detection”), and typically includes an air displacement pipettor.

Reagent cartridges, such as those described in U.S. Pat. Nos. 10,376,889; 10,406,525; 10,478,222; 10,576,474; 10,639,637 and 10,738,271 allow for particularly easy integration with liquid handling instrumentation. In some embodiments, only the air displacement pipettor is moved by the gantry and the various modules and reagent cartridge remain stationary. In alternative embodiments, an automated mechanical motion system (actuator) additionally supplies XY axis motion control or XYZ axis motion control to one or more modules and/or cartridges of the automated multi-module cell processing system. Used pipette tips, for example, may be placed by the robotic handling system in a waste repository. For example, an active module may be raised to come into contact-accessible positioning with the robotic handling system or, conversely, lowered after use to avoid impact with the robotic handling system as the robotic handling system is moving materials to other modules within the automated multi-module cell processing instrument. Alternatively, the cells may be transferred to the growth module by the user.

Alternatively, in some embodiments a gantry and/or an air displacement pump is not used; instead, in one embodiment reagents are individually connected to the bioreactor, typically via tubing or microfluidic circuits; in another embodiment, reagents may be connected to a manifold that has a single connection to the bioreactor. In some embodiments, the bioreactor is a completely closed fluidic system; that is, e.g., no pipets piercing reagent tubes and transferring liquid.

In addition, any of the growth modules described herein may reside in the same automated instrument; that is, one automated instrument may comprise two or more bioreactors each with one or more integrated cell corrals.

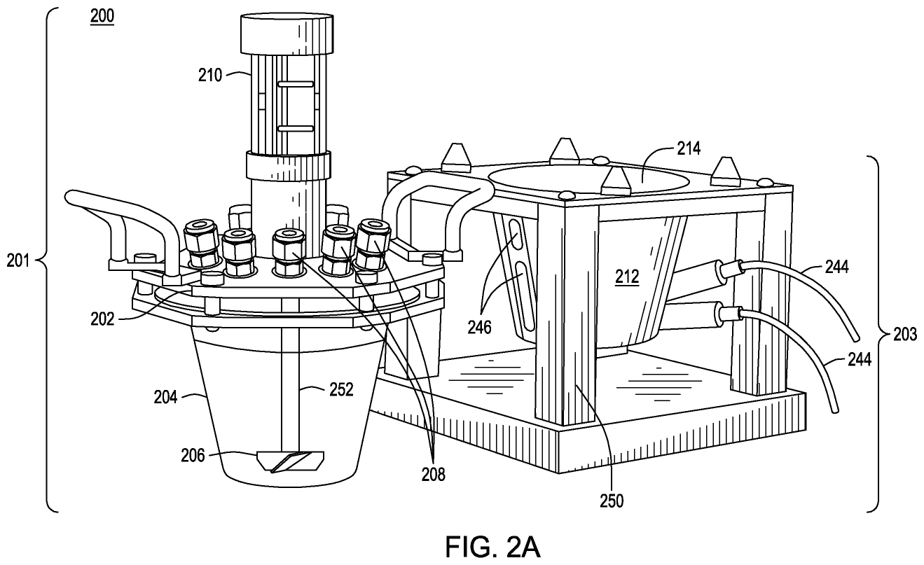

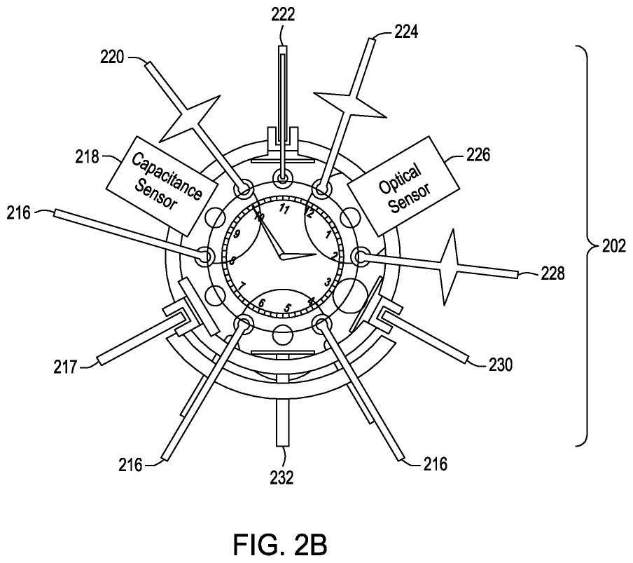

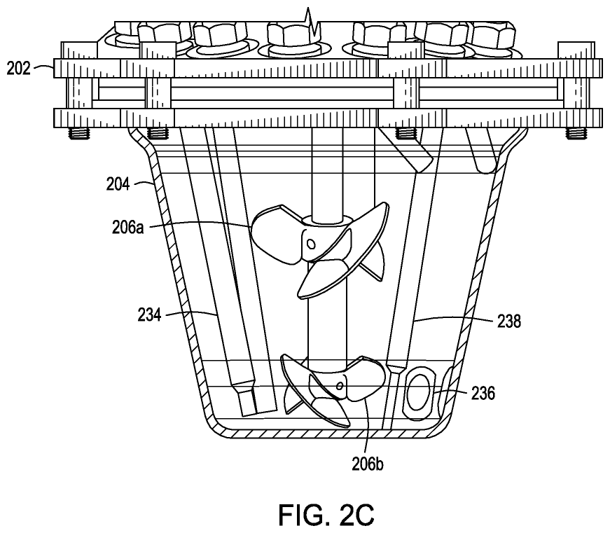

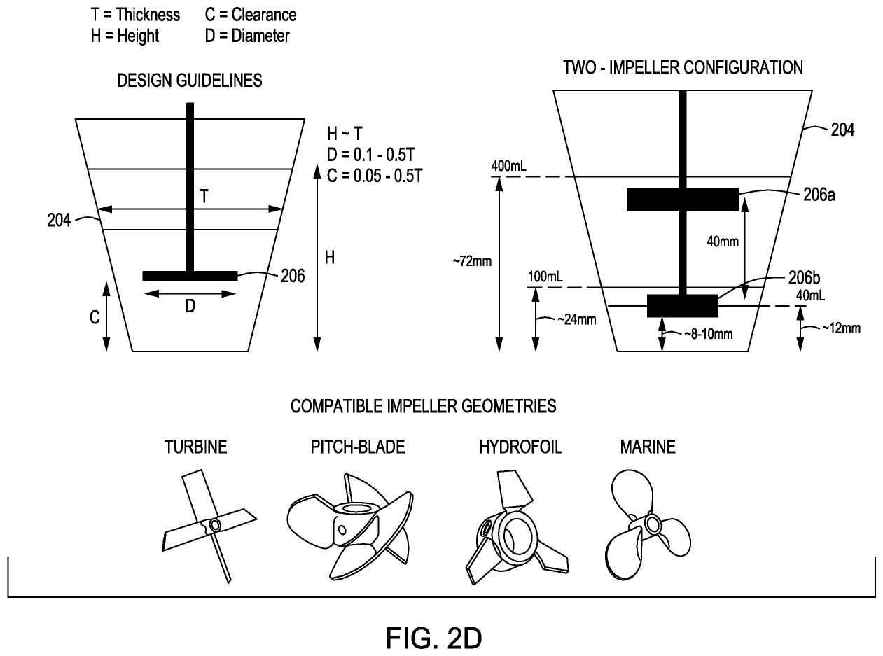

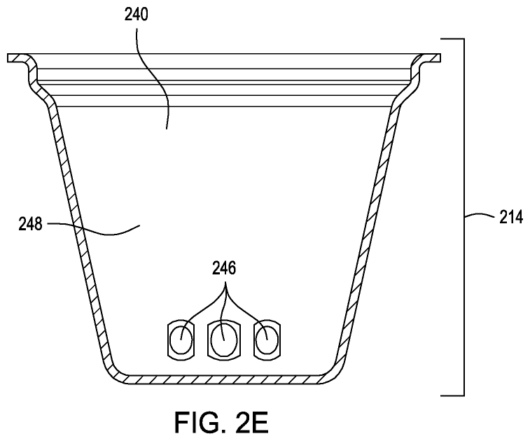

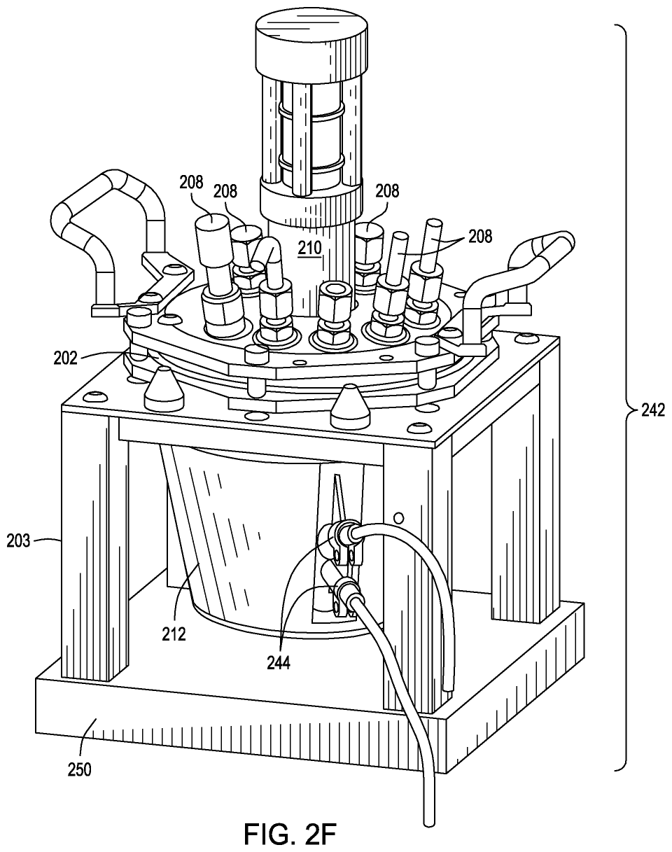

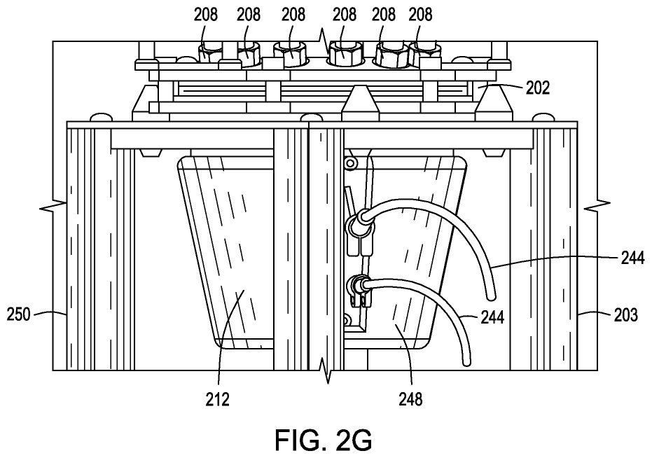

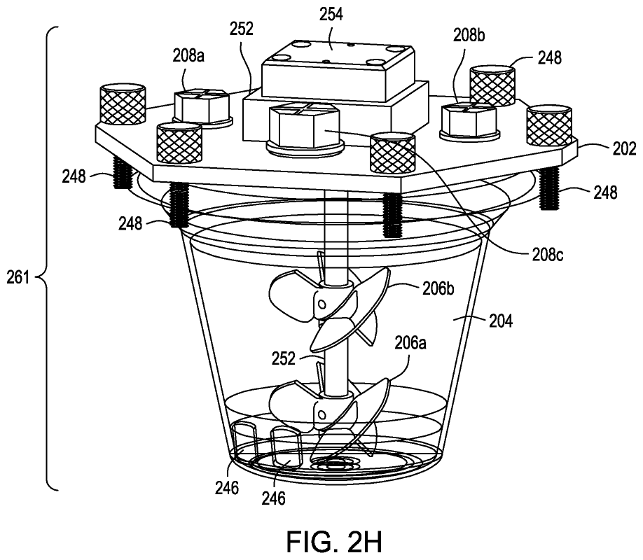

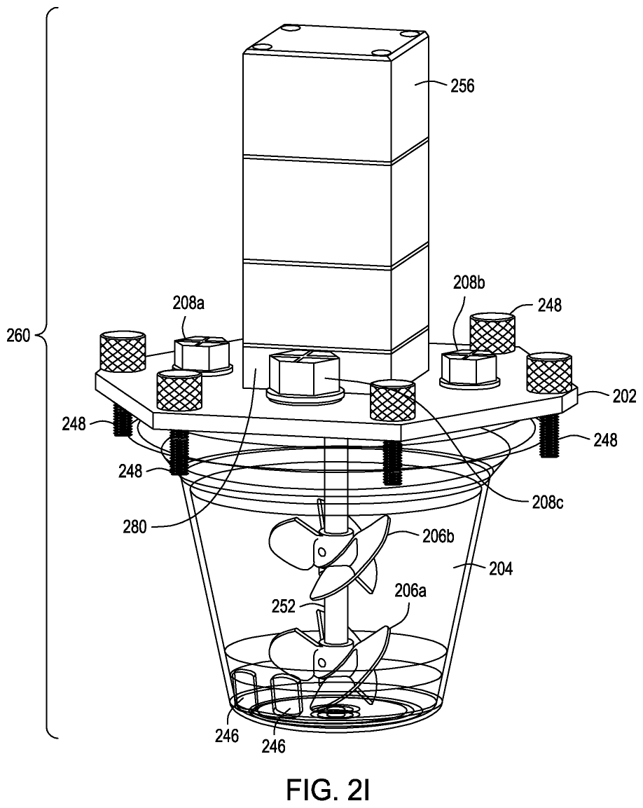

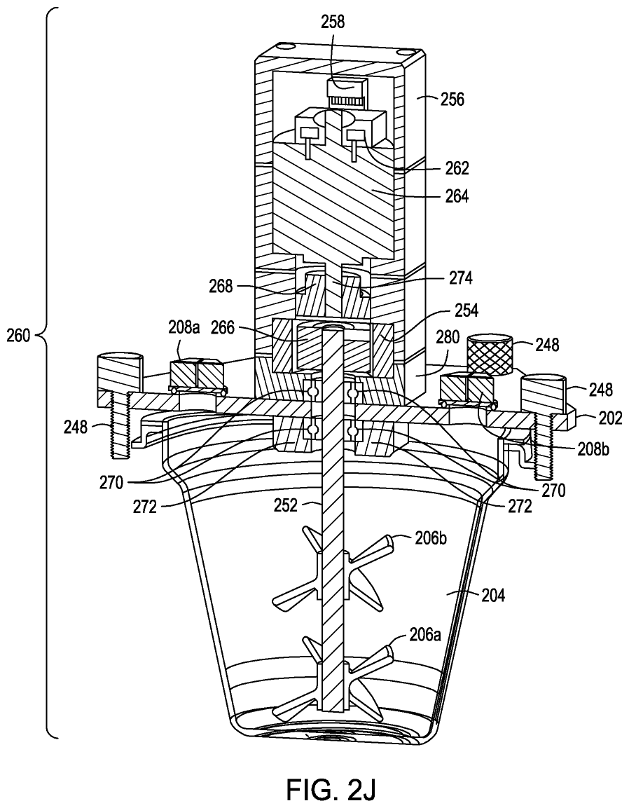

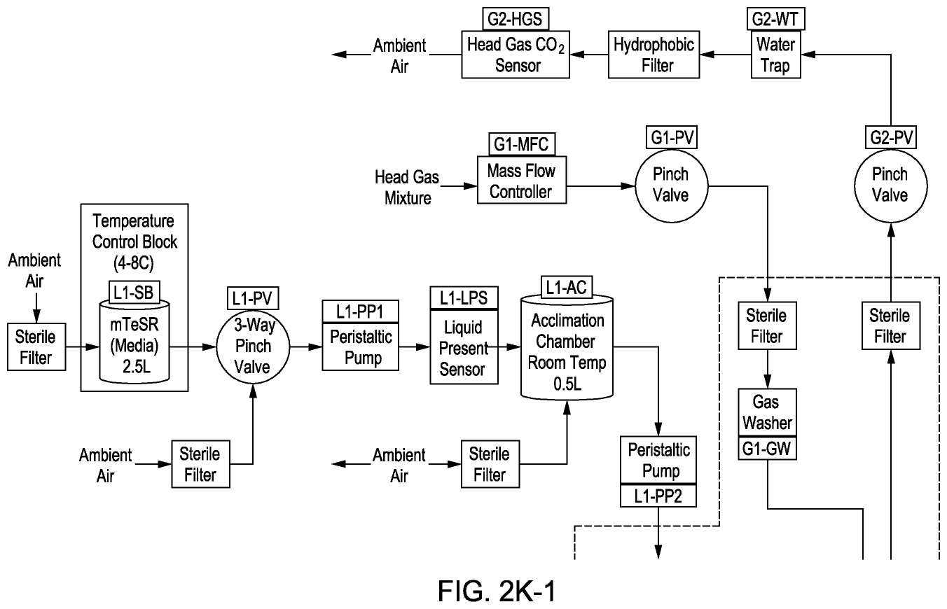

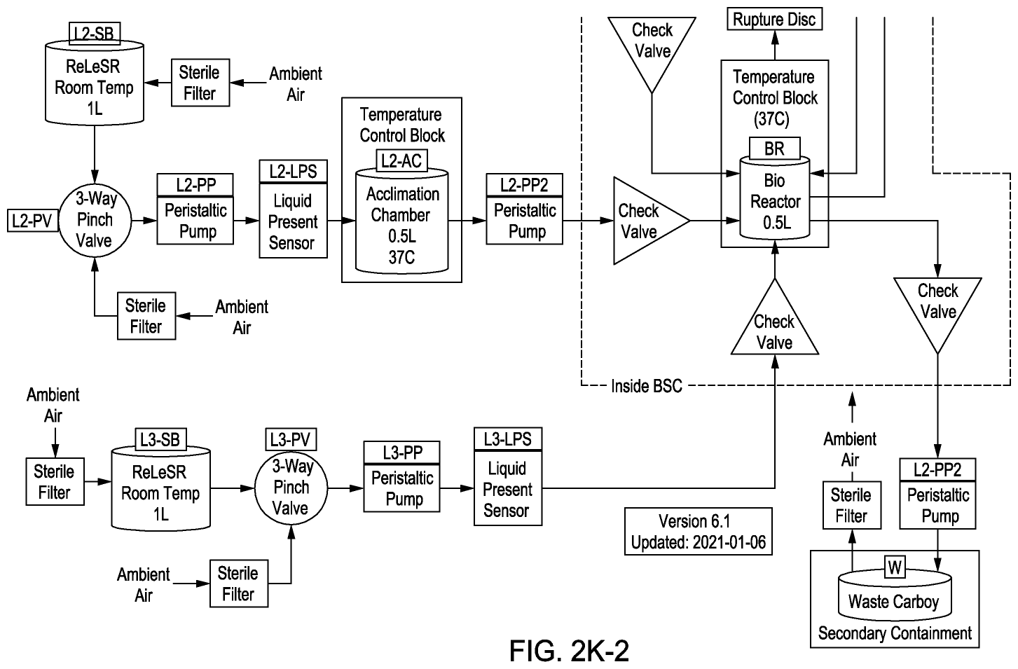

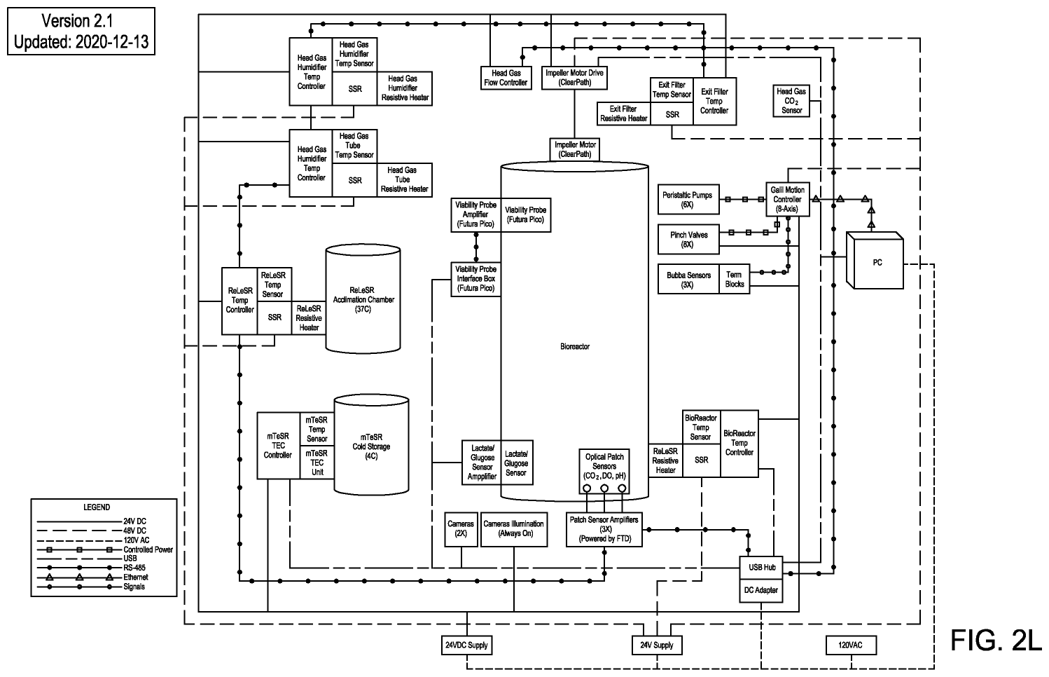

In a next step, the cells that have been grown in suspension or on microcarriers are dissociated or, if grown on microcarriers, may be dissociated from the microcarrier and/or transferred to fresh microcarriers. Dissociation is required if the cells are grown as cell aggregates or on microcarriers. In one embodiment, dissociation may be via mechanical means such as agitation or by a filter, frit or sieve. Such a filter, frit or sieve may be adapted to be part of the bioreactor module as described in relation to FIGS. 2A-2J.

As an alternative, aggregates of cells or cells and microcarriers may be dissociated by enzymes such as hemagglutinin, collagenase, dispase and trypsin, which can be added to the medium of the growing cells in the bioreactor. If the cells are grown on microcarriers, the cells can be dissociated from the microcarriers using enzymes that are typically used in cell culture to dissociate cells in 2D culture, such as collagenase, trypsin or pronase or by non-enzymatic methods including EDTA or other chelating chemicals. In a bioreactor, dissociation can be performed mechanically using, e.g., an impeller or by bubbling. Example VIII herein describes the results of cells having been detached in a bioreactor via turbulence created by an impeller.

Finally, in some methods and instruments, the population of cells after editing is enriched for edited cells by, e.g., magnetic beads, antibiotic selection, co-edit selection, or FACS sorting, all of which are described in more detail infra.

### Exemplary Embodiments for Delivery of Reagent Bundles to Mammalian Cells

FIG. 1A depicts an exemplary workflow employing microcarrier-partitioned delivery for editing mammalian cells grown in suspension where the cells are co-localized on RBMCs comprising the nucleic acids to be transfected into the cell. In a first step, the cells to be edited are grown for several passages, e.g., off instrument, to assure cell health. The cells may be grown in 2D culture, in 3D culture (if the cells are viable when grown in or adapted to 3D culture) or on microcarriers. This initial cell growth typically takes place off the automated instrument. If necessary, the cells are dissociated and added to medium in the bioreactor comprising cell growth medium such as MEM, DMEM, RPMI, or, for stem cells, mTeSR™Plus serum-free, feeder-free cell culture medium (STEMCELL Technologies Canada INC., Vancouver, BC) and cell growth microcarriers. If the cells are grown initially on microcarriers, the microcarriers are transferred to the bioreactor comprising cell growth medium such as mTeSR™Plus serum-free, feeder-free cell culture medium (STEMCELL Technologies Canada INC., Vancouver, BC) and additional microcarriers. Approximately 1e7 or 1e8 cells are transferred to the cell growth module on the automated instrument for growth.

In parallel with the off-instrument cell growth, reagent bundle microcarriers (RBMCs) are manufactured, also off-instrument. The present description provides depictions two exemplary methods for manufacturing RBMCs (see FIGS. 1C and 1D) that may be used to edit the cells in the modules and automated instruments described herein.

The cells are grown in 3D culture on microcarriers in the bioreactor for, e.g., three to four days or until a desired number of cells, e.g., 1e8, cells are present. Note that all processes in this FIG. 1A may take place in the bioreactor and cell corral. During this growth cycle, the cells are monitored for cell number, pH, and optionally other parameters. As described above, cell growth monitoring can be performed by imaging, for example, by allowing the microcarriers to settle and imaging the bottom of the bioreactor. Alternatively, an aliquot of the culture may be removed and run through a separate flow cell, e.g., in a separate module, for imaging. For example, the cell corral, in addition to being integrated with the bioreactor vessel, may be integrated with a flow cell or other device for cell counting where an aliquot of the cell culture in the cell corral may be removed and counted in the flow cell. In another alternative, the cells may express a fluorescent protein and fluorescence in the cell culture is measured or fluorescent dye may be used to stain cells, particularly live cells. This microcarrier-based workflow can be performed in the bioreactor and cell corral with most if not all steps performed in the same device; thus, several bioreactors and cell corrals may be deployed in parallel for two to many samples simultaneously. In yet another alternative, permittivity or capacitance is used to monitor cell coverage on the microcarriers. In yet another embodiment, an aliquot of cells may be removed from the bioreactor or cell corral and transported out of the instrument and manually counted on a commercial cell counter (i.e., Thermofisher Countess, Waltham, MA).

The microcarriers used for initial cell growth can be nonporous (where pore sizes are typically <20 nm in size), microporous (with pores between >20 nm to <1 μm in size), or macroporous (with pores between >1 μm in size, e.g. 20 μm). In microcarrier culture, cells grow as monolayers on the surface of nonporous or microporous microcarriers, which are typically spherical in morphology; alternatively, the cells grow on the surface and as multilayers in the pores of macroporous microcarriers. The microcarriers preferably have a density slightly greater than that of the culture medium to facilitate easy separation of cells and medium for, e.g., medium exchange and imaging and passaging; yet the density of the microcarriers is also sufficiently low to allow complete suspension of the microcarriers at a minimum stirring or bubbling rate. Maintaining a low stirring or bubbling rate is preferred so as to avoid hydrodynamic damage to the cells.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, MO, USA); Thermo Fisher (Waltham, MA, USA); Pall Corp. (Port Washington, NY, USA); GE Life Sciences (Marlborough, MA, USA); and Corning Life Sciences (Tewkesbury, MA, USA). As for the extracellular matrix, natural matrices include collagen, fibrin and vitronectin (available, e.g., from ESBio, Alameda, CA, USA), and synthetic matrices include Matrigel® (Corning Life Sciences, Tewkesbury, MA, USA), Geltrex™ (Thermo Fisher Scientific, Waltham, MA, USA), Cultrex® (Trevigen, Gaithersburg, MD, USA), biomemetic hydrogels available from Cellendes (Tubingen, Germany); and tissue-specific extracellular matrices available from Xylyx (Brooklyn, NY, USA); further, denovoMatrix (Dresden, Germany) offers screenMATRIX™, a tool that facilitates rapid testing of a large variety of cell microenvironments (e.g., extracellular matrices) for optimizing growth of the cells of interest.

Following cell growth, passaging is performed by, e.g., stopping the impeller rotation or bubbling action in the bioreactor and allowing the microcarriers to settle. In one method, the cells are removed from the microcarriers using enzymes such as collagenase, trypsin or pronase, or by non-enzymatic methods including EDTA or other chelating chemicals, and once removed from the carriers, medium is added to dilute the enzyme to inhibit enzymatic action. The dissociation procedures relating to the cell corral are described in detail infra. Once medium is added, then the cells are separated from the microcarriers by allowing the microcarriers to settle and aspirating the cells via a filtered sipper into the cell corral. The cells then may be optionally dissociated from one another via a filter, sieve or by bubbling or other agitation in the cell corral. Next, microcarriers comprising the manufactured reagent bundles (RBMCs) and the dissociated cells are combined in an appropriate medium in the growth vessel. Alternatively, instead of removing cells from the cell growth microcarriers and re-seeding on RBMCs, the cells may be transferred from the cell growth microcarriers to RBMCs via microcarrier bridge passaging either in the growth vessel in a reduced volume or in the cell corral. Bridge passaging involves allowing a new microcarrier (e.g. an RBMC) to come into physical contact with a cell-laden microcarrier, such that cells on the latter microcarrier can migrate to the RBMC.

RBMCs are not prepared on-instrument but are pre-manufactured. The microcarriers used for reagent bundles may be microporous microcarriers, which, due to the plethora of micropores, can carry a larger reagent payload per carrier diameter than nonporous or macroporous microcarriers. Preferred RBMCs are microporous, to provide increased surface area for reagent delivery, and functionalized on the surface so as to be able to bind reagents. Preferred microcarriers for RBMCs include Pierce™ Streptavidin UltraLink™ Resin, a cross-linked polyacrylamide carrier functionalized with streptavidin comprising a pore size of 50 to 100 nm; Pierce™ NeutrAvidin™ Plus UltraLink™ Resin, cross-linked polyacrylamide carrier functionalized with avidin comprising a pore size of 50 to 100 nm; and UltraLink™ Hydrazide Resin, a cross-linked polyacrylamide carrier functionalized with hydrazine comprising a pore size of 50 to 100 nm, all available from Thermo Fisher (Waltham, MA, USA); cross-linked agarose resins with alkyne, azide, photo-cleavable azide and disulfide surface functional groups available from Click Chemistry Tools (Scottsdale, AZ, USA); Sepharose™ Resin, cross-linked agarose with amine, carboxyl, carbodiimide, N-hydroxysuccinimide (NHS), and epoxy surface functional groups available from GE Health (Chicago, IL, USA).

The microcarriers are loaded with amplified editing cassettes or amplified editing plasmids, engine plasmids, nuclease or nuclease fusion proteins, mRNAs or ribonucleoproetins (RNPs) depending on, e.g., the functionalized group, via, e.g., via chemical or photo linkage or depending on a surface coating on the microcarrier, if present. RBMCs are prepared by 1) partitioning and amplifying a single copy of an editing cassette to produce clonal copies in an RBMC, or by 2) pooling and amplifying editing cassettes, followed by dividing the editing cassettes into sub-pools and “pulling down” the amplified editing cassettes with microcarriers comprising nucleic acids specific to and complementary to unique sequences on the editing cassettes. The step of sub-pooling acts to “de-multiplex” the editing cassette pool, thereby increasing the efficiency and specificity of the “pull down” process. De-multiplexing thus allows for amplification and error correction of the editing cassettes to be performed in bulk followed by efficient loading of clonal copies of the editing cassettes onto a microcarrier.

FIG. 1B depicts an exemplary option for growing, passaging, transfecting and editing iPSCs, where there is sequential delivery of clonal high copy number (HCN) RBMCs—i.e., lipid nanoparticle-coated microcarriers, where each microcarrier is coated with many copies of delivery vehicles (e.g., RNA, DNA, plasmid, or ribonucleoprotein) carrying a single clonal editing cassette—followed by bulk enzyme delivery. Note that the bioreactors and cell corrals described infra may be used for all processes. Following the workflow of FIG. 1B, first cells are seeded on the RBMCs to deliver clonal copies of nucleic acids to the cells. Again, the RBMCs are typically fabricated or manufactured off-instrument. The cells are allowed to grow and after 24-48 hours, medium is exchanged for medium containing antibiotics to select for cells that have been transfected. The cells are passaged, re-seeded and grown again, and then passaged and re-seeded, this time onto microcarriers comprising lipofectamine with the enzyme provided as a coding sequence under the control of a promoter, or as a protein on the surface of a microcarrier. As an alternative, the enzyme may be provided in bulk in solution. The enzyme is taken up by the cells on the microcarriers, and the cells are incubated and allowed to grow. Medium is exchanged as needed and the cells are detached from the microcarriers for subsequent growth and analysis.

An alternative exemplary option for the method shown in FIG. 1B comprises the steps of growing, passaging, transfecting and editing iPSCs. In this embodiment, there is simultaneous delivery of clonal high copy number (HCN) RBMCs (i.e., reagent bundle lipid nanoparticle-coated microcarriers) where each microcarrier is coated with many copies of delivery vehicles (e.g., RNA, DNA, plasmid, or ribonucleoprotein) carrying a single clonal editing cassette—and enzyme (e.g., as a coding sequence under the control of a promoter therefor, as a ribonucleoprotein complex, or as a protein). Again, the RBMCs are typically fabricated or manufactured off-instrument. Note that the integrated instrument described infra may be used for all processes. As with the workflow shown in FIG. 1B, first cells are seeded on microcarriers to grow. The cells are then passaged, detached, re-seeded, grown and detached again to increase cell number, with medium exchanged every 24-72 hours as needed. Following detachment, the cells are seeded on RBMCs for clonal delivery of the editing cassette and enzyme in a co-transfection reaction. Following transfection, the cells grown for 24-48 hours after which medium is exchanged for medium containing antibiotics for selection. The cells are selected and passaged, re-seeded and grown again. Medium is exchanged as needed and the cells are detached from the microcarriers for subsequent growth and analysis.

FIGS. 1C and 1D depict alternative methods for populating microcarriers with a lipofectamine/nucleic acid payload and cells. In the method 100a shown in FIG. 1C at top left, lipofectamine 102 and guide plasmid payloads 104 are combined and guide LNPs (lipofectamine nucleic acid payloads) 106 are formed in solution. In parallel, microcarriers 108 (“MCs”) are combined with a coating such as laminin 521 110 to foster adsorption and cell attachment. The laminin 521-coated microcarriers are then combined with the guide LNPs 106 to form partially-loaded microcarriers 112. The processes of forming RBMCs (i.e., the partially-loaded microcarriers 112 comprising the guide LNPs 106) to this point are typically performed off-instrument. In parallel and typically off-instrument, nuclease or nickase LNPs 120 are formed by combining lipofectamine 102 and nuclease or nickase mRNA 118. The nuclease or nickase LNPs 120 are combined with the partially-loaded microcarriers 112 and adsorb onto the partially-loaded microcarriers 112 to form fully-loaded RBMCs 122 comprising both the guide LNPs 106 and the nuclease or nickase LNPs 120. At this point, the mammalian cells 114 have been grown and passaged in the bioreactor and cell corral several to many times. The cells 114 populate the fully-loaded RBMCs 122, where the cells 114 then take up (i.e., are transfected by) the guide LNPs 106 and the nuclease or nickase LNPs 120, a process that may take several hours up to several days. At the end of the transfection process, transfected mammalian cells reside on the surface of the fully-loaded microcarriers 122.

As an alternative to the method 100a shown in FIG. 1C, FIG. 1D depicts method 100b which features simultaneous adsorption of the guide LNPs and the nuclease/nickase LNPs. Again, lipofectamine 102 and guide plasmid payloads 104 are combined where guide LNPs (lipofectamine nucleic acid payloads) 106 are formed in solution. In parallel, nuclease or nickase LNPs 120 are formed by combining lipofectamine 102 and nuclease or nickase mRNA 118. Also in parallel, microcarriers 108 are combined with a coating such as laminin 521 110 to foster adsorption and cell attachment. The laminin 521-coated microcarriers are simultaneously combined with both the guide LNPs 106 and the nuclease or nickase LNPs 120 to form fully-loaded microcarriers 124 where both the guide LNPs 106 and the nuclease or nickase LNPs 120 co-adsorb onto the surfect of the laminin-coated microcarriers. The processes of forming RBMCs (i.e., the fully-loaded microcarriers 124 comprising both the guide LNPs 106 and the nuclease or nickase LNPs 120) to this point are typically performed off-instrument.

At this point, the fully-loaded microcarriers 124 comprising the guide LNPs 106 and the nuclease or nickase LNPs 120 are added to medium in the bioreactor comprising the mammalian cells 114 to be transfected, optionally with additional lipofect reagent 102. The mammalian cells 114 have been grown and passaged in the bioreactor and cell corral one to many times. The cells 114 populate the fully-loaded RBMCs 124, where the cells 114 then take up (i.e., are transfected by) the guide LNPs 106 and the nuclease or nickase LNPs 120, a process that may take several hours up to several days. At the end of the transfection process, transfected mammalian cells reside on the surface of the fully-loaded microcarriers 124. In these exemplary methods, nuclease or nickase fusion mRNAs are used to form the nuclease/nickase LNPs; however, the nuclease or nickase enzymes may be loaded on to form LNPs, or gRNAs and nuclease or nickase enzymes may be loaded in the form of RNPS on the LNPs.

FIG. 1E depicts an exemplary architecture for editing cassettes to be delivered as LNPs. This architecture comprises from 5′ to 3′, primer binding sequence 1 (in this example, approximately 22 nucleotides in length); a gRNA spacer sequence (in this example, approximately 19 nucleotides in length); a gRNA scaffold sequence (in this example, approximately 76 nucleotides in length); the repair template—the nucleic acid that is designed to introduce a DNA sequence modification (insertion, deletion, substitution) into a locus by homologous recombination using nucleic acid-guided nucleases or a nucleic acid that serves as a template (including a desired edit) to be incorporated into target DNA by reverse transcriptase in a CREATE fusion editing (CFE) system (variable in length); a barcode (in this example, approximately 3 nucleotides in length), a second primer binding sequence (in this example, approximately 22 nucleotides in length); and a third primer binding sequence (in this example, approximately 22 nucleotides in length). The third primer binding sequence facilitates clonal isolation of the editing cassette from a pool of editing cassettes.

FIG. 1F depicts an exemplary workflow for creating RBMCs for pooled delivery. At top left, editing cassettes are synthesized as oligonucleotides on a substrate. The oligonucleotides are removed from the substrate and subpooled for amplification using the P3 primer binding site. The oligonucleotides are processed so as to remove oligonucleotides with errors, leaving only oligonucleotides, e.g., error-corrected editing cassettes, which were synthesized properly. The error-corrected editing cassettes are amplified to produce a pool of error-corrected editing cassettes.

In a next step, the subpooled, amplified editing cassettes are de-multiplexed using the P2 primer binding site with the number of wells needed equal to the plexity of the library of editing cassettes; e.g., 1000-plex library requires 3×384-well plates. Following demultiplexing, the editing cassettes are inserted into a vector backbone via T5 exonuclease- or Golden Gate-directed assembly and the vectors are transformed into E. coli, plated, selected and allowed to grow. Colonies are picked and plasmids are prepared. Following plasmid prep, LNPs are formed in each well and the LNPs are adsorbed onto microcarriers and pooled for transfection.

### Cell Growth and Corral Modules

### The Bioreactor

A bioreactor is used to grow cells off-instrument or to allow for cell growth and recovery on-instrument; e.g., as one module of the multi-module fully-automated closed instrument. Further, the bioreactor supports cell selection/enrichment, via expressed antibiotic markers in the growth process or via expressed antibodies coupled to magnetic beads and a magnet associated with the bioreactor. There are many bioreactors known in the art, including those described in, e.g., WO 2019/046766; 10,699,519; 10,633,625; 10,577,576; 10,294,447; 10,240,117; 10,179,898; 10,370,629; and 9,175,259; and those available from Lonza Group Ltd. (Basel, Switzerland); Miltenyi Biotec (Bergisch Gladbach, Germany), Terumo BCT (Lakewood, CO) and Sartorius GmbH (Gottingen, Germany).

FIG. 2A shows one embodiment of a bioreactor assembly 200 for cell growth, transfection, and editing in the automated multi-module cell processing instruments described herein. Unlike most bioreactors that are used to support fermentation or other processes with an eye to harvesting the products produced by organisms grown in the bioreactor, the present bioreactor (and the processes performed therein) is configured to grow cells, monitor cell growth (via, e.g., optical means or capacitance), passage cells, select cells, transfect cells, and support the growth and harvesting of edited cells. Bioreactor assembly 200 comprises cell growth vessel 201 comprising a main body 204 with a lid assembly 202 comprising ports 208, including an optional motor integration port 210 configured to accommodate a motor to drive impeller 206 via impeller shaft 252. The tapered shape of main body 204 of the growth vessel 201 along with, in some embodiments, dual impellers allows for working with a larger dynamic range of volumes, such as, e.g., up to 500 ml and as low as 100 ml for rapid sedimentation of the microcarriers.

Bioreactor assembly 200 further comprises bioreactor stand assembly 203 comprising a main body 212 and vessel holder 214 comprising a heat jacket or other heating means (not shown, but see FIG. 2E) into which the main body 204 of growth vessel 201 is disposed in operation. The main body 204 of growth vessel 201 is biocompatible and preferably transparent-in some embodiments, in the UV and IR range as well as the visible spectrum-so that the growing cells can be visualized by, e.g., cameras or sensors integrated into lid assembly 202 or through viewing apertures or slots in the main body 212 of bioreactor stand assembly 203 (not shown in this FIG. 2A, but see FIG. 2E).

Bioreactor assembly 200 supports growth of cells from a 500,000 cell input to a 10 billion cell output, or from a 1 million cell input to a 25 billion cell output, or from a 5 million cell input to a 50 billion cell output or combinations of these ranges depending on, e.g., the size of main body 204 of growth vessel 201, the medium used to grow the cells, the type and size and number of microcarriers used for growth, and whether the cells are adherent or non-adherent. The bioreactor that comprises assembly 200 supports growth of both adherent and non-adherent cells, wherein adherent cells are typically grown of microcarriers as described in detail above. Alternatively, another option for growing mammalian cells in the bioreactor described herein is growing single cells in suspension using a specialized medium such as that developed by ACCELLTA™ (Haifa, Israel). As described above, cells grown in this medium must be adapted to this process over many cell passages; however, once adapted the cells can be grown to a density of >40 million cells/ml and expanded 50-100× in approximately a week, depending on cell type.

Main body 204 of growth vessel 201 preferably is manufactured by injection molding, as is, in some embodiments, impeller 206 and the impeller shaft 252. Impeller 206 also may be fabricated from stainless steel, metal, plastics or the polymers listed infra. Injection molding allows for flexibility in size and configuration and also allows for, e.g., volume markings to be added to the main body 204 of growth vessel 201. Additionally, material from which the main body 204 of growth vessel 201 is fabricated should be able to be cooled to about 4° C. or lower and heated to about 55° C. or higher to accommodate cell growth. Further, the material that is used to fabricate the vial preferably is able to withstand temperatures up to 55° C. without deformation. Suitable materials for main body 204 of growth vessel 201 include cyclic olefin copolymer (COC), glass, polyvinyl chloride, polyethylene, polyetheretherketone (PEEK), polypropylene, polycarbonate, poly(methyl methacrylate (PMMA)), polysulfone, poly(dimethylsiloxane), cyclo-olefin polymer (COP), and co-polymers of these and other polymers. Preferred materials include polypropylene, polycarbonate, or polystyrene. The material used for fabrication may depend on the cell type to be grown, transfected and edited, and is conducive to growth of both adherent and non-adherent cells and workflows involving microcarrier-based transfection. The main body 204 of growth vessel 201 may be reusable or, alternatively, may be manufactured and configured for a single use. In one embodiment, main body 204 of growth vessel 201 may support cell culture volumes of 25 ml to 500 ml, but may be scaled up to support cell culture volumes of up to 3 L.

The bioreactor stand assembly comprises a stand or frame 250, a main body 212 which holds the growth vessel 201 during operation. The stand/frame 250 and main body 212 are fabricated from stainless steel, other metals, or polymer/plastics. The bioreactor main body further comprises a heat jacket (not seen in FIG. 2A, but see FIG. 2E) to maintain the bioreactor main body 204—and thus the cell culture—at a desired temperature. Essentially, the stand assembly can host a set of sensors and cameras to monitor cell culture.

FIG. 2B depicts a top-down view of one embodiment of vessel lid assembly 202. Growth vessel lid assembly 202 is configured to be air-tight, providing a sealed, sterile environment for cell growth, transfection and editing as well as to provide biosafety maintaining a closed system. Vessel lid assembly 202 and the main body 204 of growth vessel 201 can be sealed via fasteners such as screws, using biocompatible glues, or the two components may be ultrasonically welded. Vessel lid assembly 202 is some embodiments is fabricated from stainless steel such as S316L stainless steel but may also be fabricated from metals, other polymers (such as those listed supra) or plastics. As seen in this FIG. 2B—as well as in FIG. 2A—vessel lid assembly 202 comprises a number of different ports to accommodate liquid addition and removal; gas addition and removal; for insertion of sensors to monitor culture parameters (described in more detail infra); to accommodate one or more cameras or other optical sensors; to provide access to the main body 204 of growth vessel 201 by, e.g., a liquid handling device; and to accommodate a motor for motor integration to drive one or more impellers 206. Exemplary ports depicted in FIG. 2B include three liquid-in ports 216 (at 4 o'clock, 6 o'clock and 8 o'clock), one liquid-out port 222 (at 11 ‘clock), a capacitance sensor 218 (at 9 o'clock), one “gas in” port 224 (at 12 o'clock), one “gas out” port 220 (at 10 o'clock), an optical sensor 226 (at 1 o'clock), a rupture disc 228 at 2 o'clock, a self-sealing port 230 (at 3 o'clock) to provide access to the main body 204 of growth vessel 201; and (a temperature probe 232 (at 5 o'clock). Note that although in this embodiment there are separate liquid-in and liquid-out ports, the liquid ports may both liquid-in and liquid-out ports; that is, a liquid port may be a dedicated in or out port, or may serve both as an in port and an out port.

The ports shown in vessel lid assembly 202 in this FIG. 2B are exemplary only and it should be apparent to one of ordinary skill in the art given the present disclosure that, e.g., a single liquid-in port 216 could be used to accommodate addition of all liquids to the cell culture rather than having a liquid-in port for each different liquid added to the cell culture. Similarly, there may be more than one gas-in port 224, such as one for each gas, e.g., O₂, CO₂ that may be added. In addition, although a temperature probe 232 is shown, a temperature probe alternatively may be located on the outside of vessel holder 214 of bioreactor stand assembly 203 separate from or integrated into heater jacket 248 (not seen in this FIG. 2B, but see FIG. 2E). A self-sealing port 230, if present, allows access to the main body 204 of growth vessel 201 for, e.g., a pipette, syringe, or other liquid delivery system via a gantry (not shown). As shown in FIG. 2A, additionally there may be a motor integration port to drive the impeller(s), although other configurations of growth vessel 201 may alternatively integrate the motor drive at the bottom of the main body 204 of growth vessel 201. Growth vessel lid assembly 202 may also comprise a camera port for viewing and monitoring the cells.

Additional sensors include those that detect O₂ concentration, a CO₂ concentration, culture pH, lactate concentration, glucose concentration, biomass, and optical density. The sensors may use optical (e.g., fluorescence detection), electrochemical, or capacitance sensing and either be reusable or configured and fabricated for single-use. Sensors appropriate for use in the bioreactor are available from Omega Engineering (Norwalk CT); PreSens Precision Sensing (Regensburg, Germany); C-CIT Sensors AG (Waedenswil, Switzerland), and ABER Instruments Ltd. (Alexandria, VA). In one embodiment, optical density is measured using a reflective optical density sensor to facilitate sterilization, improve dynamic range and simplify mechanical assembly. The rupture disc, if present, provides safety in a pressurized environment, and is programmed to rupture if a threshold pressure is exceeded in growth vessel 201. If the cell culture in the growth vessel is a culture of adherent cells, microcarriers are used as described supra. In such an instance, the liquid port may comprise a filter such as a stainless steel or plastic (e.g., polyvinylidene difluoride (PVDF), nylon, polypropylene, polybutylene, acetal, polyethylene, or polyamide) filter or frit to prevent microcarriers from being drawn out of the culture during, e.g., medium exchange, but to allow dead cells to be withdrawn from the vessel. Additionally, as described infra, a liquid port may comprise a filter sipper to allow cells to be drawn into the cell corral while leaving spent microcarriers in main body 204 of growth vessel 201. The microcarriers used for initial cell growth can be nanoporous (where pore sizes are typically <20 nm in size), microporous (with pores between >20 nm to <1 μm in size), or macroporous (with pores between >1 μm in size, e.g. 20 μm) and the microcarriers are typically 50-200 μm in diameter; thus the pore size of the filter or frit in the liquid port will differ depending on microcarrier size.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, MO, USA); ThermoFisher Scientific (Waltham, MA, USA); Pall Corp. (Port Washington, NY, USA); GE Life Sciences (Marlborough, MA, USA); and Corning Life Sciences (Tewkesbury, MA, USA). As for the extracellular matrix, natural matrices include collagen, fibrin and vitronectin (available, e.g., from ESBio, Alameda, CA, USA), and synthetic matrices include MATRIGEL® (Corning Life Sciences, Tewkesbury, MA, USA), GELTREX™ (ThermoFisher Scientific, Waltham, MA, USA), CULTREX® (Trevigen, Gaithersburg, MD, USA), biomemetic hydrogels available from Cellendes (Tubingen, Germany); and tissue-specific extracellular matrices available from Xylyx (Brooklyn, NY, USA); further, denovoMatrix (Dresden, Germany) offers screenMATRIX™, a tool that facilitates rapid testing of a large variety of cell microenvironments (e.g., extracellular matrices) for optimizing growth of the cells of interest.

FIG. 2C is a side view of the main body 204 of growth vessel 201. A portion of vessel lid assembly 202 can be seen, as well as two impellers 206a and 206b. Also seen are a lactate/glucose sensor probe 234, a pH, O₂, CO₂ sensor 236 (such as a PRESENS™ integrated optical sensor (Precision Sensing GmbH, (Regensburg, Germany)), and a viable biomass sensor 238 (such as, e.g., the FUTURA PICO™ capacitance sensor (ABER, Alexandria, VA)). In some embodiments, flat regions are fabricated onto the main body 204 of growth vessel 201 to reduce optical loss, simplify spot placement and simplify fluorescent measurement of pH, dO₂, and dCO₂.

FIG. 2D shows exemplary design guidelines for a one-impeller embodiment (left) and a two-impeller embodiment (right) of the main body 204 of growth vessel 201, including four exemplary impeller configurations. The embodiment of the INSCRIPTA™ growth vessel 201 main body 204 as shown in this FIG. 2D has a total volume of 820 ml and supports culture volumes from 25 ml to 500 ml. As mentioned above, the impellers (and impeller shaft) may be injection molded or may be fabricated from stainless steel, other biocompatible metals, polymers or plastics and preferably comprised polished surfaces to facilitate sterilization. The impeller may be configured as a turbine-, pitched-blade-, hydrofoil- or marine-type impeller. In a two-impeller configuration, the impellers may be of the same type or different types. In the bioreactors described herein (the “INSCRIPTA™ bioreactors”) and used to generated the data in Examples II-IX, agitation is provided at 0-100 rpm, or 40-80 rpm, or approximately 70 rpm during cell growth (depending on the cell type being cultured); however, lower or higher revolutions per minute may be used depending on the volume of the main body 204 of growth vessel 201, the type of cells being cultured, whether the cells are adherent and being grown on microcarriers or the cells are non-adherent, and the size and configuration of the impellers. The impeller may turn in a clockwise direction, a counter-clockwise direction or the impeller may change direction (oscillate) or stop at desired intervals, particularly during cell detachment from the microcarriers and during settling of the microcarriers. Also, intermittent agitation may be applied, e.g., agitating for 10 minutes every 30 minutes, or agitating for 1 minute every 5 minutes or any other desired pattern. Additionally, impeller rpm is often increased (e.g., up to 4000 rpm) when the cells are being detached from microcarriers. Although the present embodiment of INSCRIPTA™ bioreactor utilizes one or more impellers for cell growth, alternative embodiments of the INSCRIPTA™ bioreactor described herein may utilize bubbling or other physical mixing means.

Also seen in FIG. 2D is an equation that gives a range for exemplary bioreactor dimensions based on the height (H) and thickness (T) of the main body of vessel 204. For example, D=0.25-05*T means the impeller diameter could be one quarter or one half of the main body of vessel 204 thickness, T. C is the clearance of the impeller from the bottom of the main body of vessel 204, which can be 0.15 to 0.5 times the thickness. It should be apparent to one of ordinary skill in the art given the present disclosure that these numbers are just one embodiment and the ranges may be larger. The growth vessel 201 main body 204 comprises an 8-10 mm clearance from the bottom of the main body 204 of growth vessel 201 to the lower impeller 206b and the lower impeller 206b and the upper impeller 206a are approximately 40 mm apart.

FIG. 2E is a side view of the vessel holder portion 214 of the bioreactor stand main body 212 of the bioreactor stand assembly 203. Inner surface 240 of vessel holder 214 is indicated and shown are camera or fiber optic ports 246 for monitoring, e.g., cell growth and viability; O₂ and CO₂ levels, and pH. The vessel holder portion 214 of the bioreactor stand main body 212 may also provide illumination using LED lights, such as a ring of LED lights (not shown). FIG. 2F is a side perspective view of the assembled bioreactor 242 without sensors mounted in ports 208. Seen are vessel lid assembly 202, bioreactor stand assembly 203, bioreactor stand main body 212 into which the main body 204 of growth vessel 201 (not seen in this FIG. 2F) is inserted. FIG. 2G is a lower side perspective view of bioreactor assembly 200 showing bioreactor stand assembly 203, bioreactor stand main body 212, vessel lid assembly 202 and two camera mounts 244. Surrounding bioreactor stand main body 212 is heater jacket 248.

FIG. 2H shows an alternative embodiment of a growth vessel 201 comprising a main body 204 with a lid assembly 202 comprising ports 208 (here 208a, 208b and 208c, where port 208c is larger than ports 208a and 208b), optional fiber optic ports 246, fasteners 248 used to secure the bioreactor vessel to the bioreactor stand assembly (not shown), and two impellers 206a and 206b driven by impeller shaft 252. In this FIG. 2H, there is also a lid motor coupling 280 and a lid assembly magnet cover 254. Whereas FIG. 2A shows a motor integration port 210, FIG. 2H shows the lid motor coupling 280 and a lid assembly magnet cover 254 that will be coupled with the motor (not seen in this FIG. 2H, but see FIG. 2I). Again and as in FIG. 2A, the tapered shape of main body 204 of the growth vessel 201 along with dual impellers allows for working with a larger dynamic range of volumes, such as, e.g., up to 500 ml and as low as 100 ml for rapid sedimentation of the microcarriers.

FIG. 2I shows a bioreactor/motor assembly 260 comprising growth vessel 201 shown in FIG. 2H coupled to a motor. The growth vessel 201 comprises a main body 204 with a lid assembly 202 comprising ports 208 (here 208a, 208b and 208c, where port 208c is larger than ports 208a and 208b), optional fiber optic ports 246, fasteners 248 used to secure the growth vessel to the bioreactor stand assembly (not shown), and two impellers 206a and 206b driven by impeller shaft 252. Additionally, lid motor coupling 280 is seen as is motor housing 256.

FIG. 2J is a cross section of the bioreactor/motor assembly 260 shown in FIG. 2I. Again, a bioreactor/motor assembly 260 comprising growth vessel 201 is shown. Growth vessel 201 comprises a main body 204 with a lid assembly 202 comprising ports 208 (here 208a, 208b and 208c, where port 208c is larger than ports 208a and 208b), optional fiber optic ports 246, fasteners 248 used to secure the bioreactor vessel to the bioreactor stand assembly (not shown), and two impellers 206a and 206b driven by impeller shaft 252. Also shown in this FIG. 2J is the motor comprising motor housing 256, electrical connection 258, the encoder 262, the motor 264, the magnetic component 266 of the lid 202 that couples with the magnetic component 268 of motor assembly 260, bearings 270, protective housing 272 which is configured to provide stabilization to the vessel/motor assembly and to protect the magnetic coupling from fluid, and motor shaft 274. Motors that may be employed to couple to the impeller shaft in the bioreactor include DC brushless motors, stepper motors, AC brushless, DC brushed motors and Servo motors. Other coupling configurations include jaw couplings, which are detachable, and diaphragm, disc, tire, and sleeve and gear couplings which are permanent.

FIGS. 2K-1 and 2K-2 together depict an exemplary diagram of the bioreactor fluidics. Fluidics and pneumatics are designed to establish a cell culture environment conducive for mammalian cell growth, including iPSCs. Fluidic circuits are designed to deliver and/or remove cell medium, buffers, microcarriers and additional reagents needed for growth, maintenance, selection and passaging of the cells in the automated closed culture instrument. The pneumatic circuits are designed to deliver the appropriate gas mixture and humidity for the chosen cell type, and may comprise line-in filters to prevent any contaminants from reaching the bioreactor.

FIG. 2L is a block diagram for an exemplary bioreactor control system. The control system is designed to control and automate the fluidics, pneumatics and sensor function in a closed system and without human intervention. In one embodiment, the control system is based on state-machines with a user editable state order and parameters using Json and jsonette config files. State-machines allow for dynamic control of several aspects of the bioreactor with a single computer.

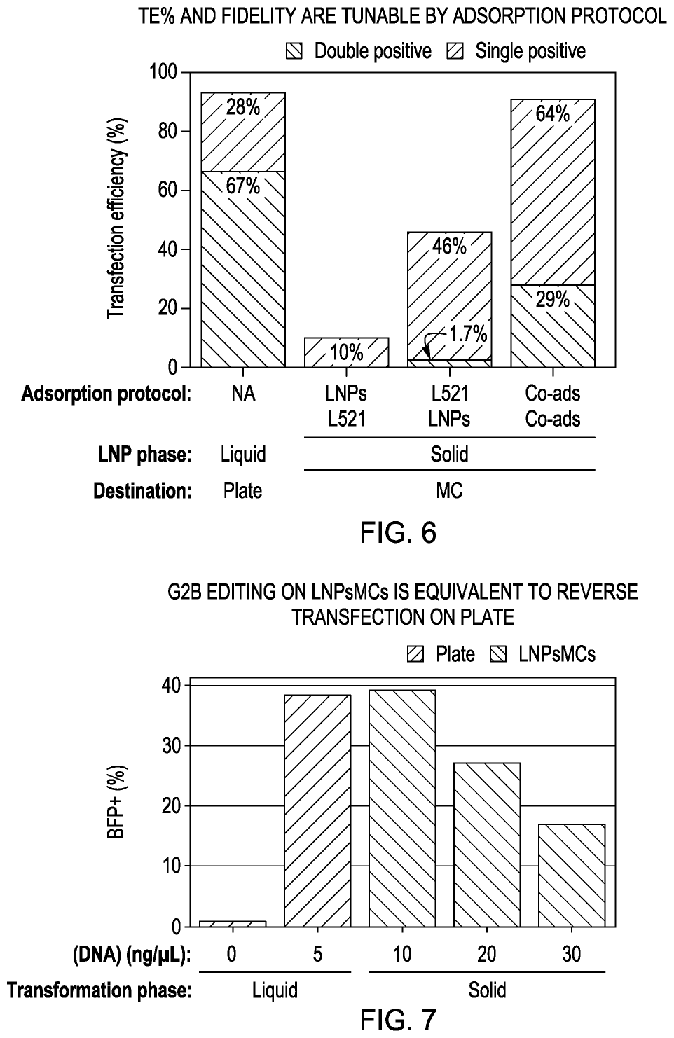

In use, the bioreactor described herein is used for cell growth and expansion-either before or after the cells are transfected in droplets—as well as for medium exchange and cell concentration. Medium/buffer exchange is in one embodiment accomplished using gravitational sedimentation and aspiration via a filter in the liquid port where the filter is of an appropriate size to retain microcarriers (see, e.g., Example VII, infra). In one embodiment used with the present bioreactor, a frit with pore size 100 μm was used and microcarriers with diameters or 120-225 μm were used in the cell culture. Sedimentation was accomplished in approximately 2-3 minutes for a 100 ml culture and 4-5 minutes for a 500 ml culture. The medium was aspirated at >100 ml/min rate. In addition to clearing the medium from the main body 204 of growth vessel 201, dead cells were removed as well. If sedimentation is used, the microcarriers do not typically accumulate on the filter; however, if accumulation is detected, the medium in the liquid port can be pushed back into main body 204 of growth vessel 201 in a pulse. In some embodiments—particularly those where sedimentation is not used—a cycle of aspiration and release (push back), aspiration and release (push back) may be performed. Experimental results show that medium exchange (aspiration) at ˜200 ml/min does not impact cell growth (see FIG. 6).

### The Cell Corral

The integrated automated instrument described herein, in addition to the bioreactor assembly, comprises a “cell corral.” The cell corral provides a temporary “warehouse” to store the cells once they have been dissociated from the microcarriers, and optionally provides a vessel in which the cells can be dissociated from one another.

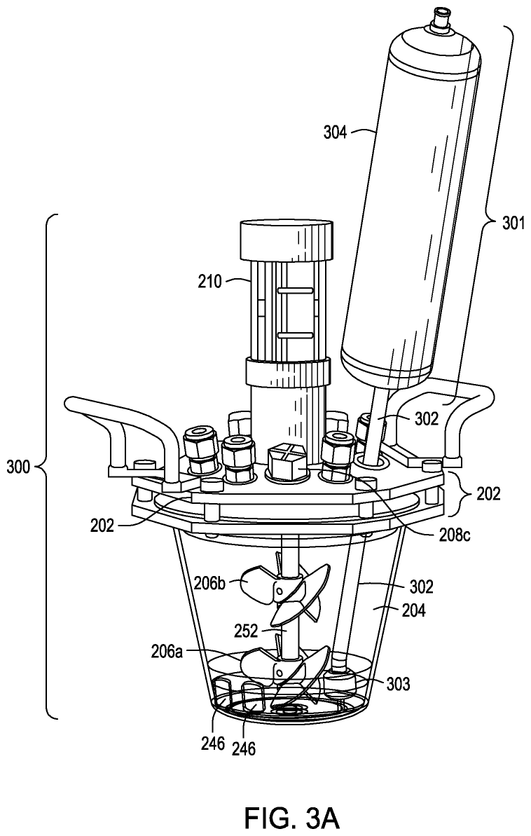

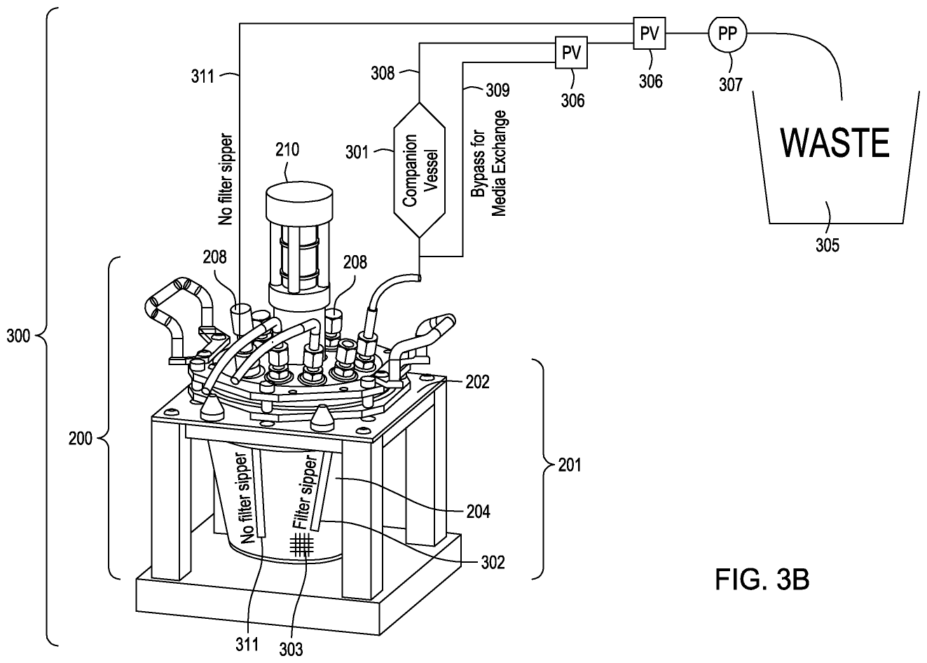

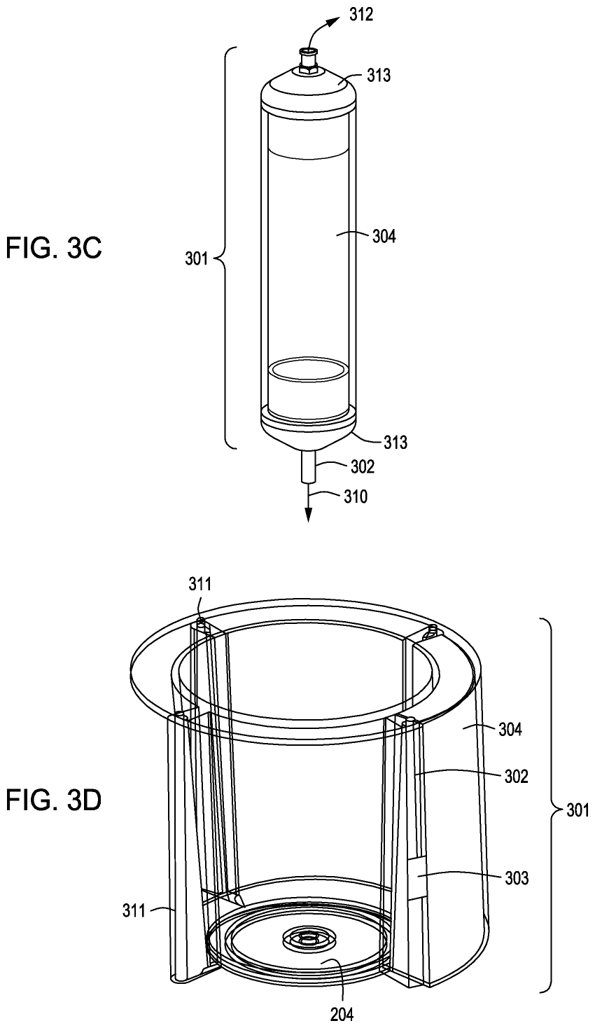

FIG. 3A shows the embodiment of a bioreactor/cell corral assembly 300, comprising the bioreactor assembly 200 for cell growth, transfection, and editing described in FIG. 2A and further comprising a cell corral 301. Bioreactor assembly 200 comprises a growth vessel 201 comprising tapered a main body 204 with a lid assembly 202 comprising ports 208, including a motor integration port 210 driving impeller 206 via impeller shaft 252. Cell corral 301 comprises a main body 304, end caps 313, where the end cap 313 proximal the bioreactor assembly 200 is coupled to a filter sipper 302 comprising a filter portion 303 disposed within the main body 204 of the bioreactor assembly 200. The filter sipper is disposed within the main body 204 of the bioreactor assembly 200 but does not reach to the bottom surface of the bioreactor assembly 200 to leave a “dead volume” for spent microcarriers to settle while cells are removed from the growth vessel 201 into the cell corral 301. The cell corral may or may not comprise a temperature or CO₂ probe: one or more cameras or LEDs or other imaging to allow for bright field cell counting (e.g., see https://www.nexcelom.com/applications/celigo/label-free-bright-fjeld-imaging live-cell/suspension-cell-counting/); an inlet for bubbling, an impeller, magnetic mixing or other mixing means; additional liquid ports for dispensing and removing wash fluid between cell passaging; and may or not be enclosed within an insulated jacket. If cells may be counted in the cell corral, the cell corral may also be configured so as to have the capability of dispensing a pre-determined number of cells (or volume of cells) into the growth vessel with the remainder of cells removed to waste.

The cell corral 301, like the main body 204 of growth vessel 201 is fabricated from any biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. Likewise, the end caps are fabricated from a biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. The cell corral may be coupled to or integrated with one or more devices, such as a flow cell (or sample tube) where an aliquot of the cell culture can be counted. Additionally, the cell corral may comprise additional liquid ports for adding medium, other reagents, and/or fresh microcarriers to the cells in the cell corral. The volume of the main body 304 of the cell corral 301 may be from 25 to 3000 mL, or from 250 to 1000 mL, or from 450 to 550 mL.

In operation, the integrated instrument 300 comprising the bioreactor 200 and cell corral 301 grows, passages, transfects, and supports editing and further growth of mammalian cells (note, the bioreactor stand assembly is not shown in this FIG. 3A). Cells are transferred to the growth vessel 201 comprising medium and microcarriers. The cells are allowed to adhere to the microcarriers. Approximately 200,000 microcarriers (laminin-521 coated polystyrene with enhanced attachment surface treatment) are used for the initial culture of approximately 20 million cells to where there are approximately 50 cells per microcarrier. The cells are grown until there are approximately 500 cells per microcarrier. For medium exchange, the microcarriers comprising the cells are allowed to settle and spent medium is aspirated via a sipper filter, wherein the filter has a mesh small enough to exclude the microcarriers. The mesh size of the filter will depend on the size of the microcarriers and cells present but typically is from 50 to 500 micrometers, or from 70 to 200 micrometers, or from 80 to 110 micrometers. For passaging the cells, spent medium is removed from the growth vessel 201, and phosphobuffered saline or another wash agent is added to the growth vessel 201 to wash the cells on the microcarriers. Optionally, the microcarriers are allowed to settle once again, and some of the wash agent is removed. At this point, the cells are dissociated from the microcarriers. Dissociation may be accomplished by, e.g., bubbling gas through the wash agent in the growth vessel 201, by increasing the impeller speed and/or direction, by enzymatic action (via, e.g., trypsin), or by a combination of these methods. In one embodiment, a chemical agent such as RelesR™ reagent (STEMCELL Technologies Canada INC., Vancouver, BC) is added to the microcarriers in the remaining wash agent for a period of time required to dissociate most of the cells from the microcarriers, such as from 1 to 60 minutes, or from 3 to 25 minutes, or from 5 to 10 minutes. Once enough time has passed to dissociate the cells, cell growth medium is added to the growth vessel 201 to stop the enzymatic reaction.

Once again, the now-spent microcarriers are allowed to settle to the bottom of the growth vessel 201 and the cells are aspirated through a filter sipper into the cell corral 301. The growth vessel 201 is configured to allow for a “dead volume” of 2 mL to 200 mL, or 6 mL to 50 mL, or 8 mL to 12 mL below which the filter sipper does not aspirate medium to ensure the settled spent microcarriers are not transported to the filter sipper during fluid exchanges. Once the cells are aspirated from the bioreactor vessel leaving the “dead volume” of medium and spent microcarriers, the spent microcarriers are aspirated through a non-filter sipper into waste. The spent microcarriers (and the bioreactor vessel) are diluted in phosphobuffered saline or other buffer one or more times, wherein the wash agent and spent microcarriers continue to be aspirated via the non-filter sipper leaving a clean bioreactor vessel. After washing, fresh microcarriers or RBMCs and fresh medium are dispensed into the bioreactor vessel and the cells in the cell corral are dispensed back into the bioreactor vessel for another round of passaging or for transfection and editing, respectively.

FIG. 3B depicts a bioreactor and cell corral assembly 300 comprising a growth vessel 201, with a main body 204, lid assembly 202 comprising a motor integration port 210, a filter sipper 302 comprising a filter 303 and a non-filter sipper 311. Also seen is a cell corral 301, fluid line 308 from the cell corral through pinch valve 306, and a line 309 for medium exchange. The non-filter sipper 311 also runs through a pinch valve 306 in waste 305. Pinch valves suitable for use in this embodiment include 2-channel pinch valves configured to pinch close flexible tubing in one channel to enable flow through the flexible tubing. Also seen is a peristaltic pump 307. In this embodiment, a peristaltic pump is employed; however, pressure-driven flow may also be suitable to manipulate fluid.

FIG. 3C depicts the cell corral 301 embodiment shown in FIGS. 3A and 3B. Cell corral 301 comprises a main body 304, end caps 313, an out port 312 to a pinch valve (not shown) and an in/out port 310 that connects to a filter sipper 302 (not shown in this FIG. 3C but see FIGS. 3A and 3B.

FIG. 3D depicts an alternative embodiment of a cell corral 301, where in this embodiment the cell corral 301 is integrated into a wall of the growth vessel main body 204. Non-filter sippers 311 are also integrated into a wall of the bioreactor vessel main body 204, as is a filter sipper 302 comprising filter 303. In yet other embodiments, the cell corral may be configured to reside underneath the growth vessel, either as an integrated subchamber or a separate, mounted chamber, and may comprise a serpentine or spiral channel for better horizontal flow control.

### Examples

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example 1: Characterization of RPMCs and iPSC Transfection Efficiency

TABLE 1

| Term | Definition |
|---|---|
| rvMC | Reagent vehicle microcarrier, or RBMC (reagent bundle microcarrier) or LNPsMCs |
| LNP | Lipid nanoparticle. The transfection reagent resulting from the incubation of the |
| | nucleic acid payload with the lipofection reagent (Lipofectamine Stem Transfection |
| | Reagent (ThermoFisher Scientific, Waltham MA). |
| L521 | Laminin 521. The extracellular matrix protein adsorbed to MCs that enables cell |
| | adherence. |
| mTeSR | iPSC growth medium (STEMCELL Technologies Canada INC., Vancouver, BC) |
| CloneR | Growth supplement added to mTeSR for the first 24 hr post-seeding to improve |
| | seeding efficiency and viability (STEMCELL Technologies Canada INC., |
| | Vancouver, BC) |
| TE | Transfection efficiency |

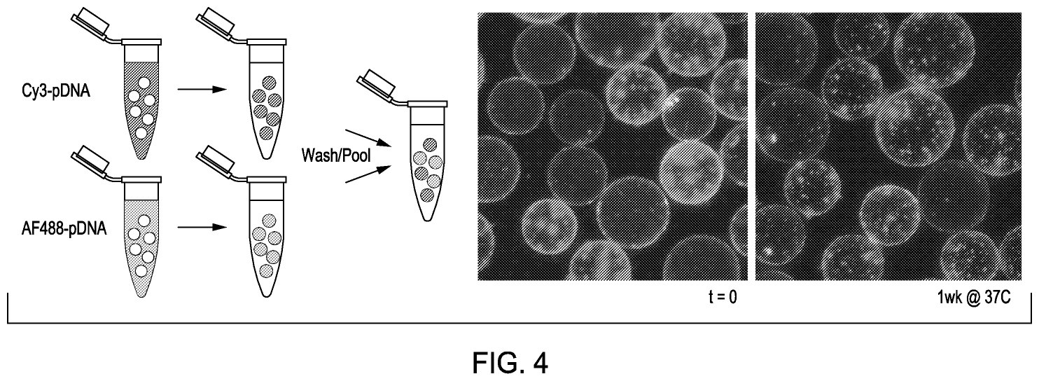

Plasmid DNA was labeled with either Cy3 or Alexa Fluor 488 using a Mirus Label IT nucleic acid labeling kit according to the manufacturer's protocol. This labeled payload was used to prepare rvMCs. First, Enhanced Attachment MCs (Corning) were autoclaved. These MCs were then washed with OptiMEM, allowed to settle, and the supernatant was aspirated. Two separate LNPs were formed by mixing a 5% v/v solution of Lipofectamine Stem in OptiMEM with a 10 ng/μL solution of the separate DNA payloads in OptiMEM and incubating at room temperature for 10 minutes. After the incubation, a stock of 100 μg/mL L521 was added to the LNP suspension to a final concentration of 10 μg/ml. The MCs were then suspended in this solution and placed in a 37° C. liquid bath. During this incubation, the MCs were suspended every five minutes by gently spinning the tube back and forth. These rvMCs were then washed three times with mTeSR+CloneR and the two batches of rvMCs with unique payloads were pooled together. Finally, the rvMCs were imaged by performing a z-scan with an epifluorescence microscope. The images in FIG. 4 are maximum intensity z-projections of these scans. The green and red puncta are LNPs adsorbed to the rvMC surface. After washing (t=0), the LNP populations are segregated between MCs. After incubating the rvMCs for one week at 37° C., puncta are still visible with minimal cross-contamination.

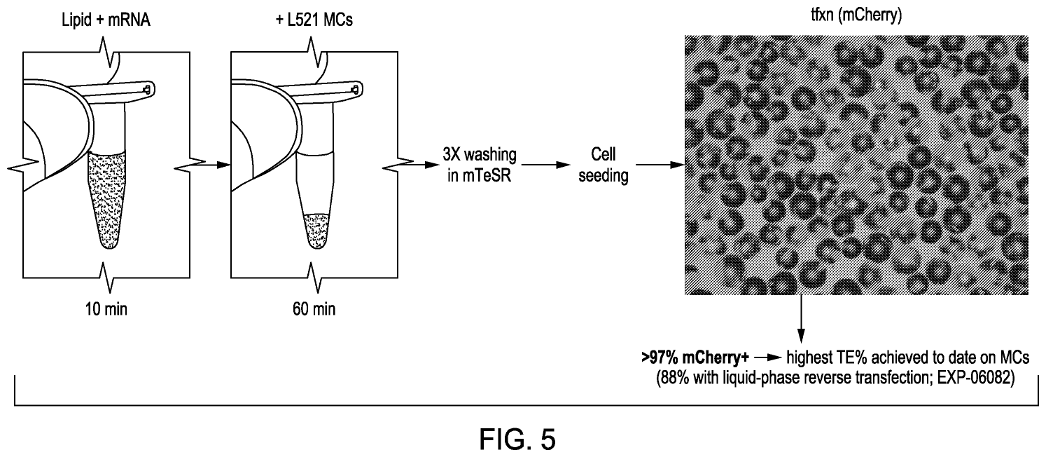

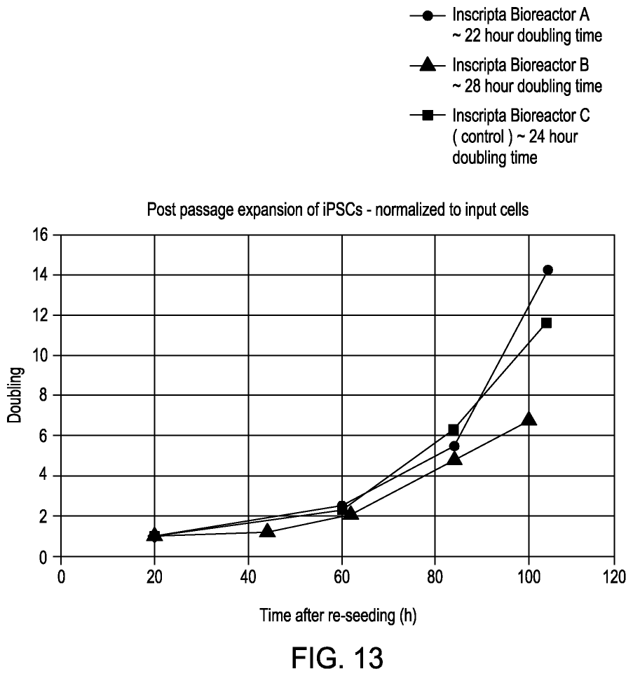

For solid phase transfection of cells with mRNA encoding for mCherry, rvMCs were prepared using a two-step assembly protocol. First, MCs were washed with DPBS+calcium+magnesium and then coated with L521 by incubating in a 10 μg/mL solution of L521 at 37° C. for 1 hour with inversions every 15 minutes. After the incubation, MCs were washed twice with OptiMEM, allowed to settle, and the supernatant was aspirated. LNPs were then assembled with the mCherry mRNA payload as described above. The LNPs form an optically-dense suspension after 10 min (FIG. 13 at left). Then, the L521-coated MCs were suspended with the LNPs and incubated in a 37° C. water bath for 60 minutes. During the incubation, the tube was gently spun every 5 minutes to resuspend the MCs. The supernatant was clear after the rvMCs settled to the bottom of the tube, indicating that the LNPs had attached to and been pulled down with the rvMCs (FIG. 5 middle). The rvMCs were washed three times in mTeSR+CloneR and iPSCs were seeded at 2×105 cells/ml. The cell and rvMC suspension was placed in a non-treated 6-well plate and shaken at 70 RPM in an incubator (37° C., 5% CO₂) overnight. Transfected cells expressed mCherry and were visualized on the surface of rvMCs using an epifluorescence microscope (FIG. 5 photograph at right). Cells were then detached from rvMCs and analyzed by flow cytometry, which revealed that the TE using rvMCs was >97%.

Pools consisting of two populations of rvMCs with distinct payloads were prepared using three adsorption protocols (see FIG. 4 and protocol described above in this Example). Instead of plasmid payloads, one population of rvMCs contained mRNA encoding for mCherry and the other contained mRNA encoding for GFP. In one adsorption protocol, MCs washed with OptiMEM were suspended in an LNP suspension prepared as described above. The MCs were incubated at 37° C. in a liquid bath for 1 hour with gentle agitation every 5 minutes. Next, the MCs were washed and resuspended in a 10 μg/ml solution of L521. The MCs were then incubated for an hour at 37° C. for 1 hour with gentle agitation every 5 minutes. Another set of MCs were first incubated with L521 following the protocol above and were then incubated with LNPs in the second incubation. In the final set of MCs, L521 and LNPs were co-adsorbed on the surfaces of MCs for 1 hour at 37° C. with gentle agitation every 5 minutes.

After the final incubation, the mCherry mRNA and GFP mRNA samples prepared by the same protocol were pooled together. Cells were seeded on the rvMCs in a non-treated 6-well plate and shaken at 70 RPM in an incubator (37° C., 5% CO₂) overnight. Cells were detached from rvMCs and were analyzed by flow cytometry. Cells that had a signal above background for both colors were considered double positive and cells with a signal above background for one of the two colors were counted as single positive. The adsorption protocol used impacted both the total transfection efficiency as well as the fraction of cross contamination (indicated by the fraction of double positive cells). The co-adsorption protocol, for example, had a total transfection efficiency of 93%, which was equivalent to the liquid-phase delivery of LNPs to cells seeded onto a Matrigel-coated plate (see FIG. 6). The number of double positive cells was lower with the solid phase delivery (29% vs. 67% with liquid-phase delivery). By adsorbing L521 first and then adsorbing LNPs, a lower transfection efficiency of 48% was achieved, but the fraction of double positive cells was only 1.7%. Thus, the performance of the rvMC solid phase delivery system is tunable by the adsorption protocol.

### Example 11: iPSC Editing Efficiency

To evaluate editing on rvMCs, a batch of rvMCs were assembled using the co-adsorption method described above. The payload was a plasmid coding for a CFE guide RNA that converts the GFP gene to a BFP gene. The expression of BFP is used as an indicator of editing in iPSCs that have a lentiviral-integrated GFP gene. Different concentrations of this DNA payload (10-30 ng/μL) were used during the initial LNP complexation before adsorption on MCs. In a separate reaction, CFE mRNA was complexed into LNPs. In this reaction, the concentration of mRNA was 25 ng/μL and the Lipofectamine Stem concentration was 5% v/v. At the time of transfection, iPSCs were seeded on rvMCs containing the plasmid payload and were co-transfected with liquid-phase LNPs containing CFE mRNA. In the control sample, cells were seeded on a Matrigel-coated plate in the presence of LNPs containing plasmid and LNPs containing CFE mRNA. With 10 ng/μL of plasmid DNA, the fraction of edited cells (as indicated by the expression of BFP) was equivalent to the fraction observed after a standard reverse co-transfection in a plate (see FIG. 7).

### Example III: Biocompatibility of Bioreactor Materials

The bioreactor and cell corral disclosed herein is one embodiment of a fully-automated, end-to-end closed instrument that does not require human intervention. Such automated, closed instruments establish and provide consistent results in a workflow and enhance uniformity of processing between “batches” while further maintaining sample integrity. Biocompatibility of bioreactor relevant materials were screened in plate cultures using conditioned media. mTeSR™Plus serum-free, feeder-free cell culture medium (STEMCELL Technologies Canada INC., Vancouver, BC) was incubated with the material of interest (i.e., stainless steel and polycarbonate) for at least 72 hours at 4° C. for conditioning the cell culture media. WTC11 iPSCs were seeded on 6-well plates and conditioned media was used to grow cells in standard incubators at 37° C., 5% CO₂ and >95% relative humidity. Control cultures were grown similarly to the tested conditions except the medium was not conditioned with any materials and the medium was kept at 4° C. for 72 hours before the start of cultures.

Cells were seeded on Matrigel coated 6-well plates (CORNING® BIOCOAT™ MATRIGEL® 6-well plates (Corning, Inc., Glendale, AZ)) and cultured with their respective conditioned (tested sample) or unconditioned media (control) and CloneR™ (STEMCELL Technologies Canada INC., Vancouver, BC) for the first 24 hours. After the first 24 hours, cell media was exchanged with fresh conditioned (tested sample) or unconditioned media (control) without CloneR, and maintained up to 72 hours where cells reached confluency. Cell counts and viabilities were assessed at 12-hours, 36-hour and 60-hour time points after lifting cells from the Matrigel CORNING® BIOCOAT™ MATRIGEL® 6-well plates (Corning, Inc., Glendale, AZ)) plates using RelesR™ reagent (following the manufacturer's instructions) (STEMCELL Technologies Canada INC., Vancouver, BC) and the cells were quantified on a NucleoCounter NC-200 (Chemometec, Allerod, Denmark) automated cell counting instrument following the manufacturer's instructions.

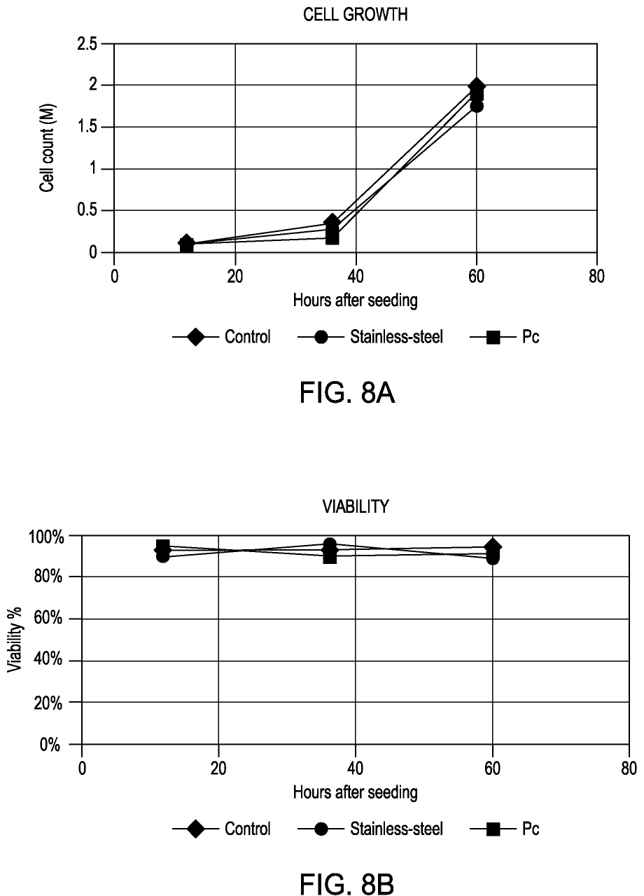

FIGS. 8A and 8B show the results of these experiments. FIGS. 8A and 8B demonstrate neither growth nor viability is impacted by the choice of materials for fabrication of the main body 204 of growth vessel 201 (polycarbonate), vessel lid assembly 202 (stainless steel), impeller 206 (stainless steel or polycarbonate), or medium exchange frit (stainless steel). All components were sterilized before conditioning.

### Example IV: Optimal Working Volume

The bioreactor described herein was tested for optimal working volume. For sensor operation, minimum optimal volume was set to 100 ml with sensor clearance at 10 mm from the bottom of the main body of the vessel. 10 million WTC11 iPSCs were seeded on 0.5 g of 10 μg/ml laminin L-521 coated Enhanced Attachment microcarriers (Corning, Inc., Glendale, AZ) in 40 ml and 100 ml mTeSRTPlus serum-free, feeder-free cell culture medium (STEMCELL Technologies Canada INC., Vancouver, BC) and CloneR (STEMCELL Technologies Canada INC., Vancouver, BC in CORNING® spinner flasks (Corning, Inc., Glendale, AZ). Impeller agitation was set to 70 rpm using a CHIMAREC™ direct stirrer (ThermoFisher Scientific, Waltham MA). A first media exchange was performed at 24 hours, and then at every 48th hour with fresh mTeSR™Plus serum-free, feeder-free cell culture medium (STEMCELL Technologies Canada INC., Vancouver, BC) (no CloneR). The cells attached to the microcarriers were quantified at 12-hour and 36-hour time points on a NucleoCounter NC-200 (Chemometec, Allerod, Denmark) automated cell counting instrument following the manufacturer's instructions. Cell counts indicated similar cell seeding efficiencies at 40 ml and 100 ml seeding volumes (data not shown).

### Example V: Assessing Growth in Bioreactor to Traditional Plating and Spinner Flask Culture

Experiments were performed to assess whether cell growth in the INSCRIPTA™ bioreactor described herein is equivalent to traditional plate and spinner flask culture conditions. Ten million WTC11 iPSCs were seeded on 0.5 g of 10 μg/ml laminin L-521 coated Enhanced Attachment microcarriers (Corning, Inc., Glendale AZ) in 100 ml mTeSR™Plus serum-free, feeder-free cell culture medium (STEMCELL Technologies Canada INC., Vancouver, BC) and CloneR (STEMCELL Technologies Canada INC., Vancouver, BC) in the INSCRIPTA™ bioreactor and in CORNING® spinner flasks (Corning, Inc., Glendale, AZ). Impeller agitation was performed at 70 rpm for both the INSCRIPTA™ bioreactor and CORNING® spinners. A control culture was also seeded on Matrigel coated 6-well plates (CORNING® BIOCOAT™ MATRIGEL® 6-well plates (Corning, Inc., Glendale, AZ)) using 500 k cells per one well. The cells were maintained at 37° C., 5% CO₂ and >95% relative humidity throughout the culture period. The first media exchange was performed at 24 hours, and then at every 48th hour with fresh mTeSRTPlus serum-free, feeder-free cell culture medium (STEMCELL Technologies Canada INC., Vancouver, BC) (no CloneR) using 100 ml for microcarrier cultures and 2 ml per well for 6-well plates. Cell counts were quantified at 12-hour, 36-hour and 60-hour time points on a NucleoCounter NC-200 (Chemometec, Allerod, Denmark) automated cell counting instrument following the manufacturer's instructions.

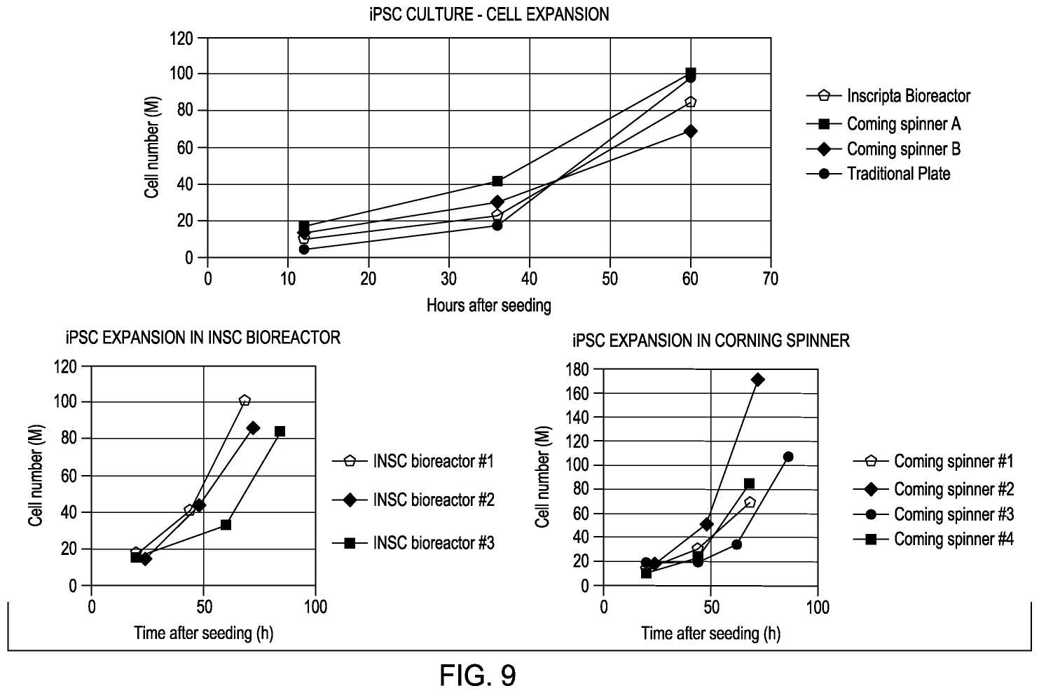

The results are shown in FIG. 9. The graph at top shows similar numbers of iPSC cells at 10, 20, 30, 40, 50, 60, and 70 hours after seeding. The graph at bottom right shows similar results were obtained for iPSC cell expansion in three different INSCRIPTA™ bioreactors. The graph at bottom left shows the results obtained for iPSC cell expansion in four different CORNING® spinner flasks. Growth curves plotted using these cell counts indicated similar cell growth curves under the conditions tested. The 6-well plate control counts were scaled assuming an initial cell seeding number of 10 million cells for comparison. Additional INSCRIPTA™ bioreactors and CORNING® spinner flasks were seeded on different days using the same methods to compare cell growth curve variations and showed similar variation across INSCRIPTA™ bioreactors and CORNING® spinners.

### Example VI: Effect of Medium Exchange

Ten million WTC11 iPSCs were seeded on 0.5 g of 10 μg/ml laminin L-521 coated Enhanced Attachment microcarriers (Corning, Inc., Glendale, AZ) in 100 ml mTeSRTPlus serum-free, feeder-free cell culture medium (STEMCELL Technologies Canada INC., Vancouver, BC) and CloneR (STEMCELL Technologies Canada INC., Vancouver, BC) in INSCRIPTA™ bioreactors and CORNING® spinner flasks. Impeller agitation was performed at 70 rpm for both the INSCRIPTA™ bioreactors and the CORNING® spinners. A 6-well plate control culture was also seeded on CORNING® BIOCOAT™ MATRIGEL® 6-well plates (Corning, Inc., Glendale, AZ) was also seeded using 500 k cells per one well. The cells were maintained at 37° C., 5% CO₂ and >95% relative humidity throughout the culture period. A first media exchange was performed at 24 hours, and then at every 48th hour with fresh mTeSRTPlus serum-free, feeder-free cell culture medium (STEMCELL Technologies Canada INC., Vancouver, BC) (no CloneR) using 100 ml for microcarrier cultures and 2 ml per well for 6-well plates.

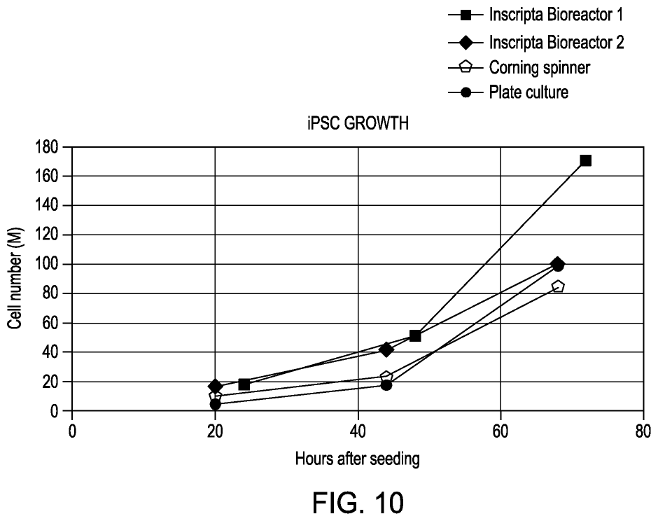

Media exchanges on the INSCRIPTA™ bioreactors were performed using a frit system as follows: Impeller agitation was stopped and the microcarriers were allowed to settle gravitationally for 5 minutes. After settling, >90% of the spent media was aspirated from the INSCRIPTA™ bioreactor through a frit connected to a peristaltic pump operating at 200 ml/min flow rate. The frit consisted of ˜100 micron pores while the microcarriers ranged from 120-225 micron in diameter. As such, microcarriers were retained in the bioreactor but spent media and dead cells were aspirated out of the bioreactor vessel. As a comparison, media exchange in CORNING® spinner flasks and 6-well plates were performed using a serological pipette connected to an aspirator (BVC Professional Aspiration System (Vacuubrand, Essex CT)). In all conditions, fresh media was added manually using a serological pipette. Cell counts were quantified at 20-hour, 44-hour and 68-hour time points on a NucleoCounter NC-200 (Chemometec, Allerod, Denmark) automated cell counting instrument following the manufacturer's instructions. The results are shown in FIG. 10. Growth curves plotted using these cell counts indicated that the media exchange approach through a frit does not have any noticeable impact on cell growth. The 6-well plate control counts were scaled assuming an initial cell seeding number of 10 million cells for comparison. During the process there was no accumulation of microcarriers on the frit in the liquid port.

### Example VII: Effect of Impeller Shear on Cell Viability and Reproducibility

Cell detachment from microcarriers may be achieved using an impeller agitation-based approach as follows: 10M cells were seeded on 0.5 g of 10 μg/ml laminin L-521 coated microcarriers (Corning, Inc., Glendale AZ), and expanded in the INSCRIPTA™ bioreactor at 100 ml mTeSR™Plus serum-free, feeder-free cell culture medium (STEMCELL Technologies Canada INC., Vancouver, BC) at 37° C., 5% CO₂, and >95% relative humidity as described above. Once the cells reached >50 million cells as determined by cell counting, the microcarriers were allowed to settle gravitationally for 5 minutes, and >90% of the spent media was aspirated. 100 ml phosphate buffered saline (PBS) was added to microcarriers for washing and aspirated after 5 minutes. 100 ml RelesR™ (STEMCELL Technologies Canada INC., Vancouver, BC) was added to the microcarriers and incubated at 37° C. for 6 minutes. After 6 minutes, >90% of the RelesR (STEMCELL Technologies Canada INC., Vancouver, BC) was aspirated and 100 ml of cell media was added to the microcarriers to quench any RelesR.

At this stage, impeller agitation was performed by rotating the impeller at 2700 rpm in the clockwise direction for 15 seconds first, and then at 2700 rpm in the counter-clockwise direction for 15 seconds. This bi-directional agitation for a total of 30 seconds duration was defined as “one round” or “one cycle”. Up to five rounds/cycles of impeller agitation was tested in terms of cell detachment efficiency. After detachment, the cell and microcarrier suspension was transferred to a conical vessel. Cells and microcarriers were separated using gravitational settling where the microcarriers settle faster than the cells due to their larger diameter. In another approach, the cell and microcarrier suspension was passed through a strainer with 100 micron mesh size (e.g., CORNING® Sterile Cell strainer-100 micron, Corning, Inc., Glendale AZ) to separate the cells from the microcarriers. As control, a 1 ml aliquot of microcarrier culture was detached using a P1000 pipette (PIPETMAN®) by passing the microcarriers through the pipette 5 times. After detachment, post detachment viability and the number of detached cells were quantified for assessing detachment efficiency.

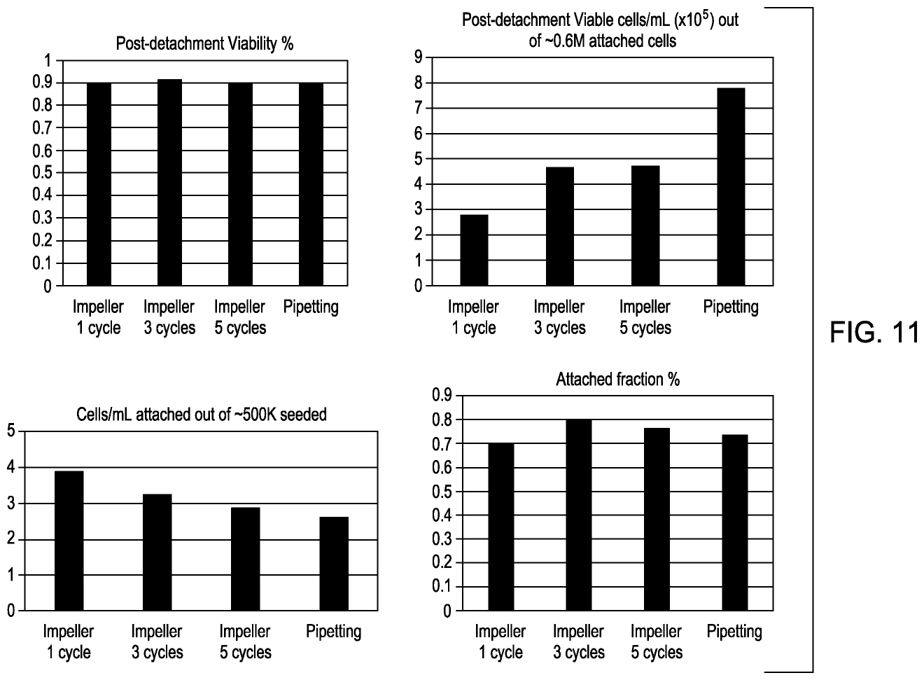

The results are shown in FIG. 11. The graph at top left of FIG. 11 shows the percent post-detachment of the cells. The graph at top right in FIG. 11 shows the number of viable cells/ml (×105) out of ˜0.6M attached cells. The graph at bottom left in FIG. 11 shows the number of cells/ml attached out of ˜500K seeded. Finally, the graph at bottom right in FIG. 11 shows the attached fraction of cells after each cycle. Note that viability remained around 90% after all of the first, third and fifth cycles. The cells were effectively detached from the microcarriers using the impeller agitation approach and showed >90% post-detachment viability after up to five rounds of impeller agitation, which was similar to the control. The re-seeding efficiency of cells detached with impeller agitation were also similar to the control case where >70% of the detached cells were able to re-seed.

Reproducibility of impeller agitation-based passaging was tested. Ten million cells were seeded on 0.5 g of 10 μg/ml laminin L-521 coated microcarriers (Corning, Inc., Glendale AZ), and expanded in the INSCRIPTA™ bioreactor in 100 ml mTeSR™Plus serum-free, feeder-free cell culture medium (STEMCELL Technologies Canada INC., Vancouver, BC) at 37° C., 5% CO₂, and >95% relative humidity as described above. Once the cells reached >50 million cells as determined by cell counting, the microcarriers were allowed to settle gravitationally for 5 minutes and >90% spent media was aspirated. 100 ml phosphate buffered saline (PBS) was added to the microcarriers for washing and was aspirated after 5 minutes. 100 ml RelesR (STEMCELL Technologies Canada INC., Vancouver, BC) were added to the microcarriers and incubated at 37° C. for 6 minutes. After 6 minutes, >90% of the RelesR was aspirated and 100 ml of cell media was added to the microcarriers to quench any RelesR.

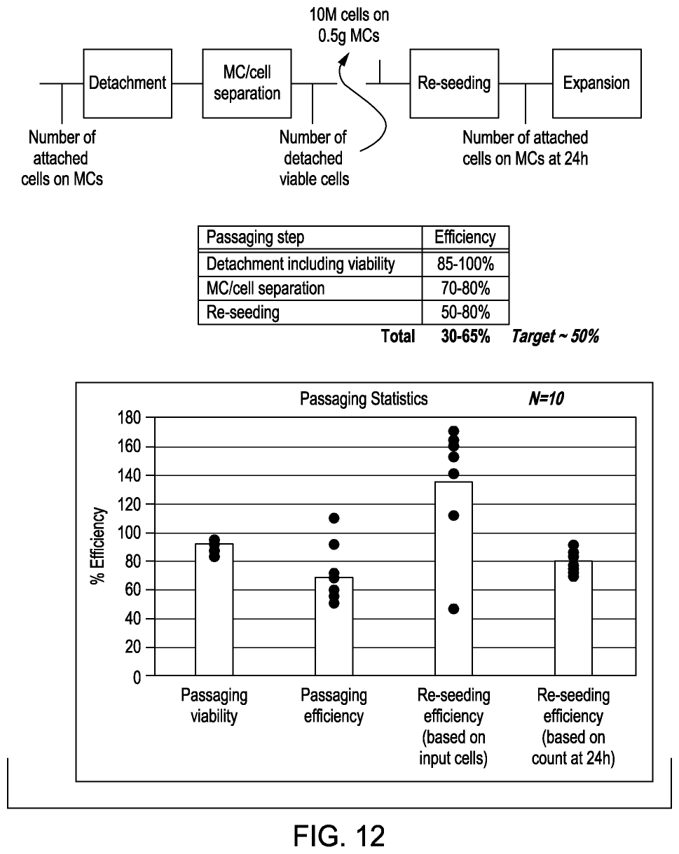

At this stage impeller agitation was performed by rotating the impeller at 2700 rpm in clockwise direction for 15 seconds first, and then at 2700 rpm in counter-clockwise direction for 15 seconds. This bi-directional agitation for a total of 30 seconds duration was defined as “one round” or “one cycle”. Three rounds/cycles of impeller agitation was used to detach the cells from microcarriers. After detachment, the cell and microcarrier suspension was transferred to a conical vessel. The cells and the microcarriers were separated using gravitational settling where the microcarriers settle faster than cells due to their larger diameter. Detached cells were re-seeded on fresh microcarriers at 10 million cells per 0.5 g of CORNING® laminin coated microcarriers (Corning, Inc., Glendale, AZ), and re-seeding efficiencies were determined based on cell counts at 24 hours after seeding. Passaging and re-seeding efficiencies are quantified and shown in the FIG. 12. FIG. 12 at top shows a simplified workflow for this process, as well as a table showing the efficiency of each step (middle), and at bottom a bar graph of passaging statistics for the indicated steps. The results indicate that impeller-based passaging is reproducible and allows for re-seeding of 30-65% of cells that were on the microcarriers prior to detachment.

### Example VIII: Cell Re-Seeding and Expansion after Impeller Passaging

Cell seeding and expansion after impeller passaging was tested. Ten million WTC11 cells were seeded on 0.5 g of 10 μg/ml laminin L-521 coated microcarriers (Corning, Inc., Glendale, AZ), and expanded in the INSCRIPTA™ bioreactor in 100 ml mTeSR™Plus serum-free, feeder-free cell culture medium (STEMCELL Technologies Canada INC., Vancouver, BC) at 37° C., 5% CO₂, and >95% relative humidity as described above. Once the cells reached >50 million cells as determined by cell counting, the impeller passaging protocol was implemented as described above. After detachment, 10M detached cells were re-seeded on 0.5 g of fresh laminin coated microcarriers (Corning, Inc., Glendale, AZ) and expanded as described above. As a control, an INSCRIPTA™ bioreactor was seeded with cells detached from T75 flasks detached using standard protocols. Cell counts were quantified at 20-hour, 44-hour and 68-hour time points on a NucleoCounter NC-200 (Chemometec, Allerod, Denmark) automated cell counting instrument following the manufacturer's instructions. The results are shown in FIG. 13. FIG. 13 is a graph of triplicate results demonstrating that cell seeding and expansion are unaffected by impeller-shear passaging.

### Example IX: Ability of Cells to Maintain Stemness

The ability of the iPSCs to retain stemness during culture and passaging was tested. Ten million cells were seeded on 0.5 g of 10 μg/ml laminin L-521 coated microcarriers (Corning, Inc., Glendale, AZ), and expanded in an INSCRIPTA™ bioreactor in mTeSR™Plus serum-free, feeder-free cell culture medium (STEMCELL Technologies Canada INC., Vancouver, BC) at 37° C., 5% CO₂, and >95% relative humidity as described above. Once the cells reached >50 million cells as determined by cell counting, the impeller passaging protocol was implemented and 10M detached cells were re-seeded onto fresh 0.5 g laminin coated microcarriers (Corning, Inc., Glendale, AZ). This process was repeated two more times and the cells were stained after final detachment using antibodies (BIOLEGEND®, San Diego, CA) specific to three stemness expression markers (TRA-1-60, OCT-3/4 and SOX-2) following the manufacturer's instructions, followed by analysis using flow cytometry (BD FACSMelody™) (Becton Dickinson, Inc., Franklin Lakes, NJ). Cells grown and impeller passaged on the INSCRIPTA™ bioreactors showed expression of stemness markers similar to the cells grown on Matrigel (CORNING® BIOCOATT MATRIGEL® 6-well plates (Corning, Inc., Glendale, AZ)) and laminin coated plates (CORNING® BIOCOAT™ laminin plates (Corning, Inc., Glendale, AZ)).

Stemness antibody staining was performed in the following manner, with the equipment and materials listed in Table 2:

TABLE 2
Foxp3/Transcription Factor Fixation/Permeabilization Concentrate and Diluent, ThermoFisher
Scientific, cat. # 00-5521-00
eBioscience ™ Flow Cytometry Staining Buffer, ThermoFisher Scientific, cat. # 00-4222-26
Anti-SOX2 (Brilliant Violet 421): Biolegend, cat. # 656114
Anti-OCT3/4 (Alexa488): Biolegend, cat. # 653706
Anti-TRA-1-60 (PE-Cy7): Biolegend, cat. # 330620
Anti-CD44 (PE-Cy5): ThermoFisher Scientific, cat. # 15-0441-82
Anti-CD13 (PE-Cy7): Biolegend, cat. # 301712
Anti-NESTIN (Alexa488): Biolegend, cat. # 656812
Anti-SSEA4 (V450): BD Biosciences, cat. # 561156
FACSMelody ™ flow cytometer (Becton Dickinson, Inc., Franklin Lakes, NJ)

In a first step, a single-cell suspension was prepared and centrifuged 5 minutes at 200×g. The cells were then washed in an appropriate volume of DPBS and centrifuged again for 5 minutes at 200×g. The supernatant was discarded and the pellet was vortexed to dissociate the pellet. Fresh Foxp3 fixation/permeabilization working solution (ThermoFisher Scientific, Waltham MA) was prepared by mixing one part Foxp3 fixation/permeabilization concentrate with three parts Foxp3 fixation/permeabilization diluent and 1 ml was added to each tube and each tube was then vortexed. The vortexed cells and fixation/permeabilization working solution were incubated for 30-60 minutes in the dark at room temperature. A 1× working solution of permeabilization buffer was prepared by mixing one part 10× permeabilization buffer with nine parts dH₂O and 2 ml was added to each sample. The cells were centrifuged at 400-600× g for 5 minutes at room temperature and the supernatant was discarded. The cell pellet was resuspended in 1× permeabilization buffer for a total volume of approximately 100 μl. The cells were diluted so that there were no more than 10,000 cells/μl, and 1M cells were transferred to a fresh tube. The appropriate amount of directly-conjugated antibody was dispensed into each tube. The cells were incubated for >30 minutes in the dark at room temperature. Two ml of 1× permeabilization buffer was added to each tube and the samples were centrifuged at 400-600× g for 5 minutes at room temperature and the supernatant was discarded. The stained cells were suspended in flow cytometry staining buffer.

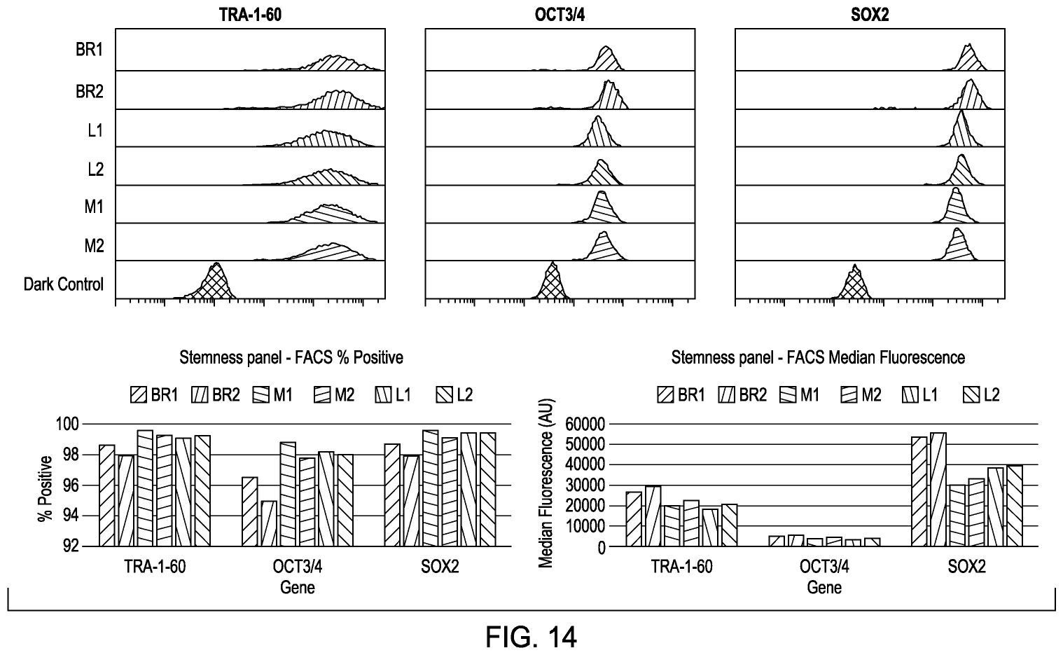

The results are shown in FIG. 14. FIG. 14 at top are histograms showing the fluorescent expression distribution measured via flow cytometry of the cell population for individual stemness marker expression. The x-axis shows the fluorescence signal and the y-axis shows cell count. BR1 indicates results for INSCRIPTA™ bioreactor 1, BR2 indicates results for INSCRIPTA™ bioreactor 2 (replicate), L1 indicates CORNING® BIOCOATT laminin plates (Corning, Inc., Glendale, AZ), L2 indicates CORNING® BIOCOAT™ laminin plates (Corning, Inc., Glendale, AZ) (replicate), M1 indicates CORNING® BIOCOAT™ MATRIGEL® 6-well plates (Corning, Inc., Glendale, AZ), and M2 indicates CORNING® BIOCOAT™ MATRIGEL® 6-well plates (Corning, Inc., Glendale, AZ) (replicate). A dark control was used for comparison where the cells in one well from the M1 6-well plate are prepared as the experimental cells but were not stained with antibodies. Looking at the graph at bottom left of FIG. 14, note that the percent of cells positive for the TRA-1-60 and SOX2 cell surface markers was similar across culture conditions. Cell surface marker OCT3/4 was a little lower (94-96%) in the cells grown in the INSCRIPTA™ bioreactors than in the laminin plates (98%) and in the MATRIGEL® plates (98%). The graph at right of FIG. 22 shows the median fluorescence obtained for each of TRA 1-60, OCT3/4 and SOX2 markers for each bioreactor, laminin plate and MATRIGEL® plate replicate.

### Example X: Ability of Cells to Maintain Differentiation Potential

To test whether cells grown in the INSCRIPTA™ bioreactor would retain differentiation potential, ten million cells were seeded on 0.5 g of 10 μg/ml laminin L-521 coated microcarriers (Corning, Inc., Glendale AZ), and expanded in INSCRIPTA™ Bioreactor in 100 ml mTeSR™Plus serum-free, feeder-free cell culture medium (STEMCELL Technologies Canada Inc., Vancouver, BC) at 37° C., 5% CO₂, and >95% relative humidity as described above. Once the cells reached >50 million cells as determined by cell counting, the impeller passaging protocol as described above in Example VII was implemented and 10M detached cells were re-seeded onto 0.5 g fresh laminin coated microcarriers. This process was repeated two more times, and after the final detachment the cells were seeded on 12-well plates for trilineage differentiation using a commercial protocol (STEMDIFF™ Trilinage Differentiation Kit, STEMCELL Technologies Canada Inc., Vancouver, BC). After trilineage differentiation, the cells from each lineage were stained with antibodies specific to markers specific to that lineage (available from BIOLEGEND®, San Diego CA and Miltenyi Biotec, San Diego, CA) following the manufacturer's instructions. The cells grown and impeller-passaged on the INSCRIPTA™ bioreactors showed expression of lineage-specific markers similar to the cells grown on Matrigel and laminin coated plates.

The tri-lineage differentiation antibody staining protocol was performed in the following manner, with the equipment listed in Table 4 and the antibodies listed in Table 3:

TABLE 3
Foxp3/Transcription Factor Fixation/Permeabilization Concentrate and Diluent, ThermoFisher
Scientific, cat. # 00-5521-00
eBioscience ™ Flow Cytometry Staining Buffer, ThermoFisher Scientific, cat. # 00-4222-26
FACS staining buffer (2% FBS, 1 mM EDTA, 0.5% BSA)
FACS buffer (2% FBS, 1 mM EDTA)
FACSMelody ™ flow cytometer (Becton Dickinson, Inc., Franklin Lakes, NJ)

TABLE 4

| | | Antibody | Catalog | | | | |
|---|---|---|---|---|---|---|---|
| Marker | Cell Type | Link | # | Conjugate | Isotype | Isotype | Conc. |
| CXCR4 | Mesoderm | BioLegend | 306518 | BV421 | Mouse | BioLegend | 1:200 |
| | | | | | IgG2a, κ | | |
| NCAM1 | Mesoderm | BioLegend | 362510 | PE-Cy7 | Mouse | BioLegend | 1:200 |
| | | | | | IgG1, κ | | |
| Brachyury | Mesoderm | SantaCruz | sc- | AF488 | Mouse | BioLegend | 1:25 |
| | | | 374321 | | IgG2b, κ | | |
| | | | AF488 | | | | |
| Nestin | Ectoderm | BioLegend | 656808 | BV421 | Mouse | BioLegend | 1:400 |
| | | | | | IgG2a, κ | | |
| Otx-2 | Ectoderm | Miltenyi | 130-121- | Vio B515 | recombinant | Miltenyi | 1:100 |
| | | | 202 | | hs IgG1 | | |
| PAX6 | Ectoderm | Miltenyi | 130-123- | PE | recombinant | Miltenyi | 1:400 |
| | | | 250 | | hs IgG1 | | |
| CXCR4 | Endoderm | BioLegend | 306518 | BV421 | Mouse | BioLegend | 1:400 |
| | | | | | IgG2a, κ | | |
| SOX17 | Endoderm | Miltenyi | 130-111- | Vio B515 | recombinant | Miltenyi | 1:600 |
| | | | 147 | | hs IgG1 | | |
| FOXA2 | Endoderm | BD | 561589 | PE | Mouse | BD | >1:20 |
| | | Biosciences | | | IgG1, κ | Biosciences | |

A single-cell suspension was prepared by lifting cells with TrypLET SELECT (ThermoFisher Scientific, Waltham, MA, USA) and was centrifuged for 5 minutes at 200× g. The cells were washed in DPBS and centrifuged a second time. The cells were fixed with a Foxp3 kit (ThermoFisher Scientific, Waltam MA) according to the manufacturer's instructions. Following incubation at room temperature in the dark for 30-60 minutes, 1 ml Foxp3 fixation/permeabilization working solution was added. Each sample contained ≤10M cells. A 1× working solution of permeabilization buffer was prepared by mixing one part of 10× Permeabilization Buffer with nine parts of distilled water and 2 ml of 1× permeabilization buffer was added to each tube. The samples were centrifuged at 400-600× g for 5 minutes at room temperature. The supernatant was discarded and the pellet was resuspended in residual volume of 1× permeabilization buffer for a total volume of approximately 100 μl. The cells were diluted so that there were no more than 10,000 cells/μl in a 96-well V- or U-bottom plate. A master mix of antibodies per cell lineage in FACS staining buffer was prepared. Approximately 500,000 cells were stained in 50 μl of staining solution. The cells were incubated on ice in the dark for at least 30 minutes. 150 μl of FACS buffer was added to each well. The cells were then centrifuged at 500×g for 5 minutes at room temperature and the supernatant was discarded. The cells were resuspended in FACS buffer and analyzed by a flow cytometer on the FACSMelody™ flow cytometer.

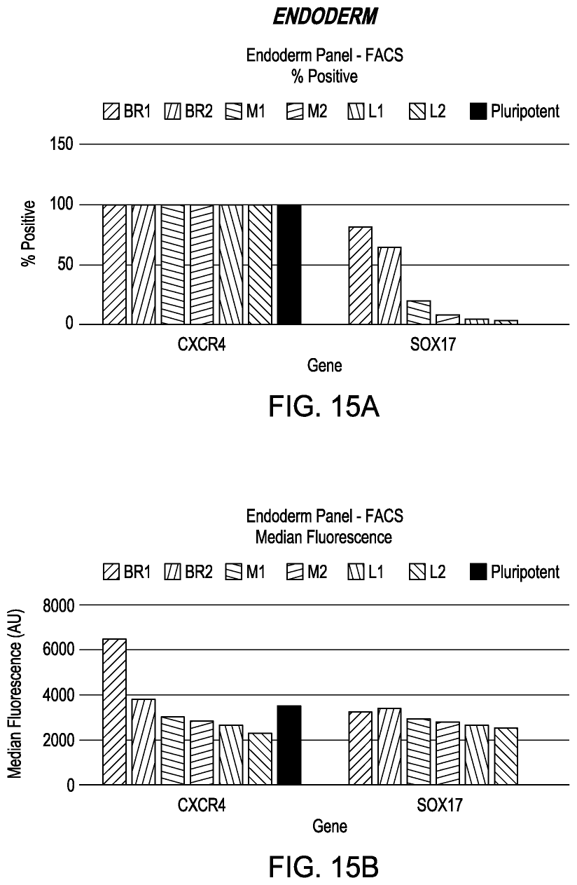

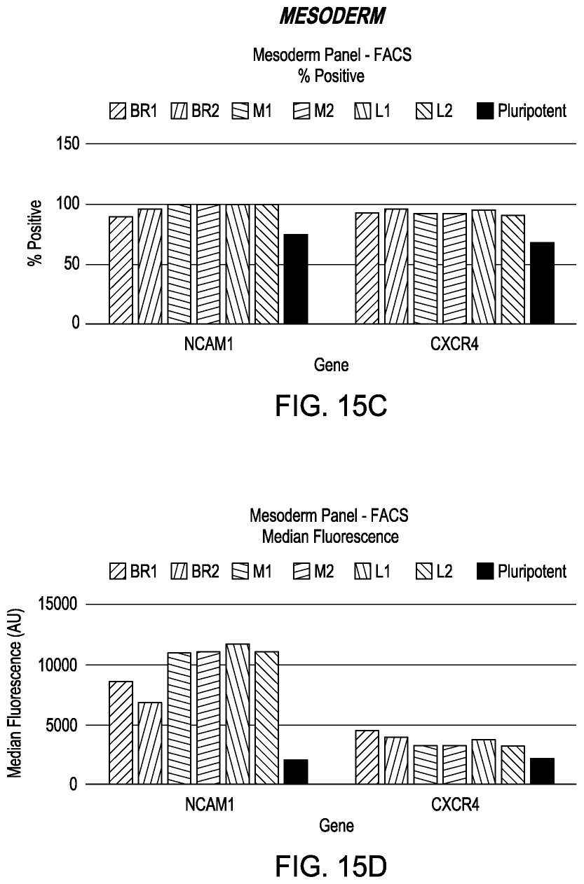

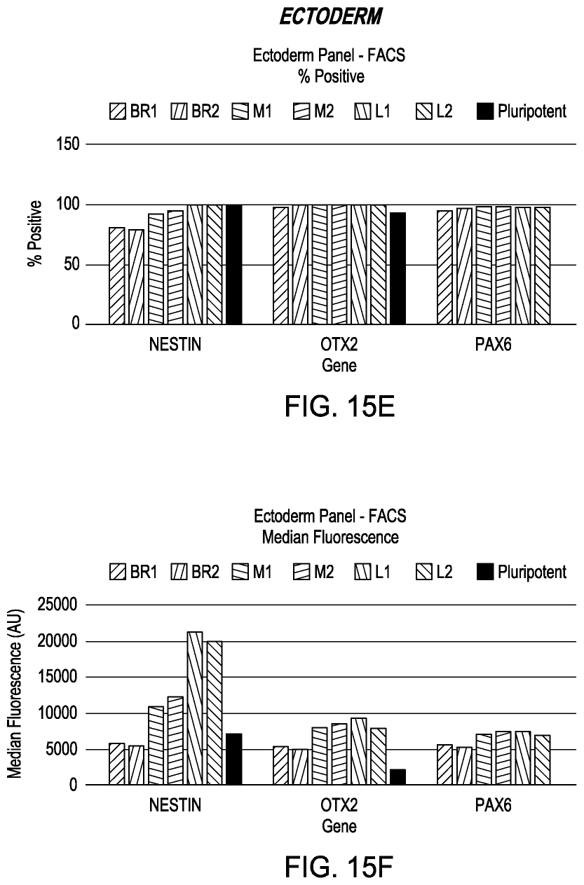

The results are shown in FIG. 15A-15F. FIGS. 15A, 15C and 15E are bar graphs showing % positive cells for endoderm markers CXCR4 and SOX17; mesoderm markers NCAM1 and CXCR4; and ectoderm markers NESTIN, OTX2 and PAX6. FIGS. 15B, 15D and 15F are bar graphs showing median fluorescence obtained for the endoderm, mesoderm and ectoderm markers. BR1 indicates results for INSCRIPTA™ bioreactor 1, BR2 indicates results for INSCRIPTA™ bioreactor 2 (replicate), L1 indicates CORNING® BIOCOAT™ laminin plates (Corning, Inc., Glendale, AZ), L2 indicates CORNING® BIOCOAT™ laminin plates (Corning, Inc., Glendale, AZ) (replicate), M1 indicates CORNING® BIOCOAT™ MATRIGEL® 6-well plates (Corning, Inc., Glendale, AZ), and M2 indicates CORNING® BIOCOATT MATRIGEL® 6-well plates (Corning, Inc., Glendale, AZ) (replicate). Note that the cells grown in the bioreactors maintain differentiation potential roughly equivalent to cells grown in the laminin plates and MATRIGEL® plates. A pluripotent control was used, where the pluripotent control were cells that were not differentiated using the STEMDIFF medium (STEMDIFF™ Trilinage Differentiation Kit, STEMCELL Technologies Canada Inc., Vancouver, BC) but were maintained in mTeSRPlus medium (STEMCELL Technologies Canada INC., Vancouver, BC).

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, 16.

1. An integrated instrument for growing, passaging and editing cells comprising:
a bioreactor comprising:
a growth vessel comprising a tapered main body, a lid assembly comprising ports, at least one driving impeller, and an impeller shaft, wherein there is at least two liquid ports, wherein at least one of the liquid ports comprises a filtered sipper; at least one gas-in port; at least one gas-out port; at least one rupture disc; and at least one sensor port; and wherein the lid assembly makes an air-tight fitting on the tapered main body; and
a bioreactor stand assembly comprising a frame, a stand main body disposed in the frame, wherein the stand main body accommodates the tapered main body of the growth vessel during operation, and wherein the stand main body comprises a heating element to heat the tapered main body; and
a cell corral comprising a main body configured to store cells and fluidically coupled to the bioreactor tapered main body via the liquid port comprising the filtered sipper.

2. A method of growing cells and passaging the cells in an integrated instrument, comprising the steps of:
providing an integrated instrument comprising:
a bioreactor comprising a growth vessel comprising a tapered main body, a lid assembly comprising ports, at least one driving impeller, and an impeller shaft, wherein there is at least two liquid ports, wherein at least one of the liquid ports comprises a filtered sipper and at least one of the liquid ports comprises a non-filtered sipper; at least one gas-in port; at least one gas-out port; at least one rupture disc; and at least one sensor port; and wherein the lid assembly makes an air-tight fitting on the tapered main body; and a bioreactor stand assembly comprising a frame, a stand main body disposed in the frame, wherein the stand main body accommodates the tapered main body of the growth vessel during operation, and wherein the stand main body comprises a heating element to heat the tapered main body; and
a cell corral comprising a main body configured to store cells and fluidically coupled to the bioreactor tapered main body via the liquid port comprising the filtered sipper;
providing microcarriers comprising a cell adhesion agent in cell growth medium to the growth vessel;
providing cells to the growth vessel;
allowing the cells to adhere to the microcarriers;
growing the cells on the microcarriers;
dissociating the cells from the microcarriers;
allowing the microcarriers to settle on a bottom of the growth vessel;
aspirating the cells into the cell corral via the liquid port comprising the filtered sipper;
aspirating the microcarriers into waste via the liquid port comprising the non-filtered sipper;
washing the growth vessel;
adding fresh medium and microcarriers to the growth vessel, wherein the microcarriers comprise a cell adhesion agent;
transferring the cells in the cell corral to the growth vessel via the liquid port comprising the filtered sipper; and
allowing the cells to adhere to the microcarriers.

3. The method of claim 2, comprising the further steps of:
growing the cells on the microcarriers;
dissociating the cells from the microcarriers;
allowing the microcarriers to settle on a bottom of the growth vessel;
aspirating the cells into the cell corral via the liquid port comprising the filtered sipper;
aspirating the microcarriers into waste via the liquid port comprising the non-filtered sipper;
washing the growth vessel;
providing cell growth medium and reagent bundle microcarriers to the tapered main body of the growth vessel, wherein each reagent bundle microcarrier comprises clonal copies of editing cassettes, a selection marker, a coding sequence for a nucleic acid-guided nuclease and a lipofection agent;
allowing the cells to attach to and grow on the reagent bundle microcarriers;
providing conditions for the editing cassettes to transfect the cells;
selecting for transfected cells via the selection marker;
dissociating the cells from the reagent bundle microcarriers;
allowing the reagent bundle microcarriers to settle in the bottom of the growth vessel; and
aspirating the cells into the cell corral via the liquid port comprising the filtered sipper.
